(19) United States
(12) Patent Application Publication
(10) Pub. No.: US 2022/0028549 A1
(43) Pub. Date: Jan. 27, 2022

(54) Analysis and Visualization of Microbial Communities

(71) Applicant: Genentech, Inc., South San Francisco, CA (US)

(72) Inventors: Joseph Nathaniel PAULSON, San Francisco, CA (US); Janina REEDER, Belmont, CA (US)

(73) Assignee: Genentech, Inc., South San Francisco, CA (US)

(21) Appl. No.: 17/379,857

(22) Filed: Jul. 19, 2021

Related U.S. Application Data
(60) Provisional application No. 63/054,859, filed on Jul. 22, 2020.

(51) Int. Cl. G16H 50/20; G06F 8/38

(52) U.S. Cl. CPC G16H 50/20; G06F 8/38

(57) ABSTRACT

In one embodiment, a software tool for performing microbiome analyses including accessing microbiome samples, generating a user interface associated with the software tool, wherein the user interface comprises input fields, and wherein each of the input fields corresponds to one or more of a phenotype or a feature associated with the microbiome samples, receiving user inputs to one or more of the input fields via the user interface of the software tool, generating a visualization comprising analysis results associated with the microbiome samples at the user interface, wherein the analysis results are generated based on the user inputs, and outputting an exportable report and software code comprising the generated visualization.

### PRIORITY

This application claims the benefit, under 35 U.S.C. § 119(e), of U.S. Provisional Patent Application No. 63/054859, filed 22 Jul. 2020, which is incorporated herein by reference.

### FIELD OF THE INVENTION

The present disclosure relates to a software tool and methods to analyze and visualize the composition and population structure of microbial communities.

### BACKGROUND

High-throughput DNA sequencing technologies, including targeted marker-gene sequencing, are providing unprecedented insight into the composition and population structure of microbial communities. Although it may seem that DNA sequencing should reduce many scientific questions to simply counting the organisms' representative sequences, there are subtle but significant challenges associated with sampling, counting, and representational statistics. As a consequence, many microbiome-specific analytical pipelines and workflows have been developed.

High-throughput sequencing of microbial communities provides a tool to characterize associations between the host microbiome and health status (i.e., healthy or disease phenotypes), to detect pathogens, and to identify the interplay of an organism's microbiome with the built environment. Large amounts of microbiome sample data may be available, but significant effort is required for researchers to effectively analyze them, which limits the utility of these rich data resources.

Potentially pathogenic or probiotic bacteria can be identified by detecting significant differences in their distribution across healthy and disease populations, thereby making the analysis of differential abundance critical. Similar issues are encountered in the attempt to correlate microbiome composition with environmental factors. Although methods for comparing whole communities are commonly used in this context, there is a need for tools that discern taxon-specific associations in marker-gene surveys.

While tools and packages exist to answer many of the questions asked of this exciting new data type, analyses remain inaccessible to researchers without the right skill set. A desire exists to make microbiome analyses and studies more accessible to researchers who lack programming and statistical skills. Effective integrative and interactive visual and statistical tools to analyze the large-scale microbiome sample data can greatly increase the value of these data for researchers.

### SUMMARY OF PARTICULAR EMBODIMENTS

Herein is provided a software tool and methods to analyze and visualize the composition and population structure of microbial communities.

In particular embodiments, a software tool and methods may be used for microbiome data analyses. The software tool and methods may be used to design microbiome studies, understand phenotype interactions, parse variables, and facilitate microbiome study analysis. The software tool and methods may be used to predict subjects' prognosis, predict subjects' responsiveness to various treatments, identify a treatment effective for an individual subject, and/or assign subjects into appropriate arm within a clinical trial. In particular embodiments, microbiome sample data may be accessed at a computing system via the software tool. The software tool may generate a user interface comprising phenotype and feature selection options. A user of the software tool may provide user input comprising phenotype and feature selections of the microbiome sample data. Upon receiving the user input, the software tool may generate a visualization from the user input and the microbiome sample data and an exportable report and software code comprising the generated visualization as output. In particular embodiments, the software tool may further aggregate counts of features in the microbiome sample data based on the user input and generate the visualization from the aggregated counts. In particular embodiments, the software tool may additionally generate an output of an indication that a subject is eligible for a clinical trial in testing a medical treatment for a particular medical condition.

In particular embodiments, the software tool may access a plurality of microbiome samples. The software tool may then generate a user interface. The user interface may comprise one or more input fields. In particular embodiments, each of the input fields may correspond to one or more of a phenotype or a feature associated with the plurality of microbiome samples. The software tool may then receive, via the user interface, one or more user inputs to one or more of the input fields. In particular embodiments, the software tool may generate, at the user interface, a visualization comprising one or more analysis results associated with the plurality of microbiome samples. The one or more analysis results may be generated based on the one or more user inputs. The software tool may further output an exportable report and software code comprising the generated visualization.

### BRIEF DESCRIPTION OF THE DRAWINGS

FIG. 1 illustrates an example workflow of the software tool.

FIG. 2 illustrates an example initial interface of the software tool.

FIG. 3A illustrates an example user interface for an overview of quality-control (QC) plots.

FIG. 3B illustrates an example user interface for a bar plot.

FIG. 4A illustrates an example user interface for a bar plot with color options.

FIG. 4B illustrates an example user interface for a bar plot with phenotype options.

FIG. 5A illustrates an example user interface for phenotype information.

FIG. 5B illustrates an example user interface for adjusting the plot datatype.

FIG. 6 illustrates an example user interface for an overview of microbiome features.

FIG. 7 illustrates an example user interface for data aggregation.

FIG. 8A illustrates an example user interface for selecting the feature level for intra-sample analysis.

FIG. 8B illustrates an example user interface for selecting analysis parameters for intra-sample analysis.

FIG. 9A illustrates an example user interface for relative abundance.

FIG. 9B illustrates an example user interface for relative abundance with facet options.

FIG. 9C illustrates an example user interface for selecting plot options for relative abundance.

FIG. 10 illustrates an example user interface for feature abundance with plot options.

FIG. 11 illustrates an example user interface for alpha diversity with plot options.

FIG. 12A illustrates an example user interface for beta diversity.

FIG. 12B illustrates an example user interface for beta diversity with principle component analysis and permutational multivariate analysis of variance.

FIG. 13 illustrates an example user interface for abundance heatmap.

FIG. 14A illustrates an example user interface for feature correlation.

FIG. 14B illustrates an example user interface for phenotype correlation with plot options.

FIG. 15A illustrates an example user interface for differential analysis.

FIG. 15B illustrates an example user interface for differential analysis with a specific level.

FIG. 16 illustrates an example user interface for longitudinal analysis.

FIG. 17A illustrates an example user interface for survival analysis.

FIG. 17B illustrates another example user interface for survival analysis.

FIG. 18A illustrates an example user interface for report generation via the software tool.

FIG. 18B illustrates an example report generated by the software tool.

FIG. 19 illustrates an example diagram flow using the software tool for microbiome analyses.

FIG. 20 illustrates an example method for microbiome analyses.

FIG. 21 illustrates an example method for survival analysis.

FIG. 22 illustrates an example of a computing system.

### DESCRIPTION

In particular embodiments, the software tool may provide microbiome-specific analyses in a user-friendly interface and guided workflow, thus removing the prerequisite of having software programming skills for users to study microbiome data. The software tool may also suggest analyses or visualizations based on data input for analysis. This may help users in understanding large-scale data sets and readily direct users to insights of the microbiome data of these data sets. In addition, the software tool may provide simulations of experiments including mixed-effect models, spline models, and various use cases. In particular embodiments, the software tool may be a software package, software application, or web interface. As an example and not by way of limitation, the software tool may include an R package, which allows a user to perform and visualize microbiome analytical workflows either through the command line or an interactive Shiny application included with the package. In addition to applying common analytical workflows, the software tool may enable automated analysis report generation. The software tool may have several technical advantages. One technical advantage may include addressing the needs of both computational scientists by combining different analysis methods in one package, but also the needs of bench scientists with a limited coding background. Another technical advantage may include furthering microbiome data analysis by suggesting parameters or feature to compare and providing simulations of data at various time points. Another technical advantage may include delivering a set of powerful and well-designed interactive visualizations, which distinguishes it from conventional work that may be dependent on command lines or may not contain all the visualization capabilities as featured in the software tool disclosed herein.

In particular embodiments, the software tool may access a plurality of microbiome samples. The software tool may then generate a user interface. The user interface may comprise one or more input fields. In particular embodiments, each of the input fields may correspond to one or more of a phenotype or a feature associated with the plurality of microbiome samples. The software tool may then receive, via the user interface, one or more user inputs to one or more of the input fields. In particular embodiments, the software tool may generate, at the user interface, a visualization comprising one or more analysis results associated with the plurality of microbiome samples. The one or more analysis results may be generated based on the one or more user inputs. The software tool may further output an exportable report and software code comprising the generated visualization.

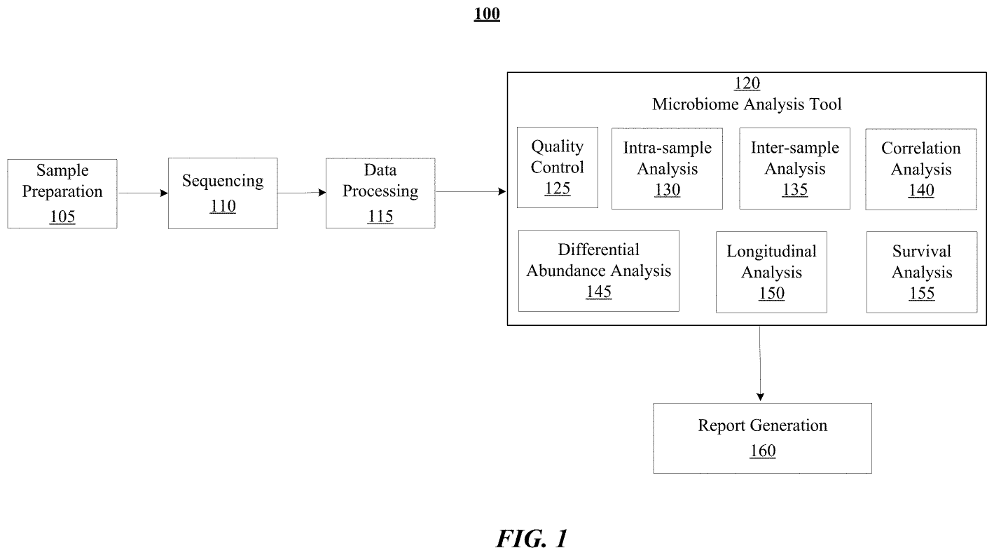

In particular embodiments, a computing system may preprocess a plurality of microbiome samples for microbiome analyses to be performed by a software tool. The computing system may then input the processed microbiome samples to the software tool. The computing system may then use the software tool to perform quality control on the processed microbiome samples. In particular embodiments, the computing system may use the software tool to perform one or more microbiome analyses based on one or more user inputs. The computing system may further generate an exportable report comprising analysis results associated with the one or more microbiome analyses. FIG. 1 illustrates an example workflow 100 of the software tool. As indicated in FIG. 1, the workflow 100 may start with sample preparation 105, sequencing 110, and data processing 115. As an example and not by way of limitation, the data processing 115 may comprise post-processing of reads. Then taxonomy and associated metadata may be uploaded into the software tool 120. In particular embodiments, the software tool 120 may comprise a plurality of modules for different microbiome analyses. As an example and not by way of limitation, the modules may comprise a module for quality control 125, a module for intra-sample analysis 130, a module for inter-sample analysis 135, a module for correlation analysis 140, a module for differential abundance analysis 145, a module for longitudinal analysis 150, and a module for survival analysis 155. In particular embodiments, the software tool 120 may further generate a report using report generation 160 based on the microbiome analyses, which may be downloadable. As an example and not by way of limitation, the generated report may comprise one or more of time, introduction, descriptions of data loading and preparation, analysis at genus level, or a visualization of an analysis.

In particular embodiments, an initial step of to perform an analysis within the software tool 120 may be to pre-process data as desired following the understanding of sampling characteristics. Initial visualizations may help in quality control (QC) 125 and understanding of sequencing depth and feature detection characteristics. A next step may comprise selecting (or receiving a selection of) a taxonomy level, and aggregating data at the chosen level for downstream analyses.

In particular embodiments, intra-sample analysis 130 may comprise functions focused on investigating the microbial composition within a sample or a group of samples. The software tool 120 may provide user options and visualizations to explore the relative abundance, feature abundance or alpha diversity. In particular embodiments, inter-sample analysis 135 may focus on differences between samples or groups of samples through heatmaps of variable or differentially abundant operational taxonomic units (OTUs) or features and beta diversity calculations. Beta diversity distance metrics may vary within the software package and include Bray-Curtis. In particular embodiments, feature correlation analysis 140 of the software tool 120 may provide scatterplots of abundance between features and correlation statistics which may be faceted by phenotypes. A test for significance within each facet may be performed. Phenotype correlation 140 may also be provided, in which the relationship between a feature and a continuous phenotype may be analyzed. In particular embodiments, the software tool 120 may perform differential abundance analysis 145 using DESeq2, Kruskal-Wallis, limma, or a zero-inflated log normal model. DESeq2 and limma are methods for comparisons in microarray and RNA-sequencing data that have been adapted for microbiome data. Kruskal-Wallis is a non-parametric test for any differences in distribution between groups. The zero-inflated log normal model is implemented in the metagenomeSeq package to account for zero-inflation in microbiome data. In particular embodiments, the software tool 120 may provide longitudinal analysis 150, including the option to visualize feature abundances across specific levels of a phenotype, such as dates or tissue types. Optionally, a separate phenotype may be used to link a common data point across the different levels with lines. In particular embodiments, the software tool 120 may additionally provide survival analysis 155, including the option to visualize survival analysis of sample data. The survival analysis 155 may help users understand the samples that were lost track of or the samples that haven't had an event (e.g., death). In particular embodiments, the software tool 120 may further report or output selected analyses and export them in files with different formats such as HTML, PDF, Word document, or PowerPoint with R markdown code for reproducibility and adjustments.

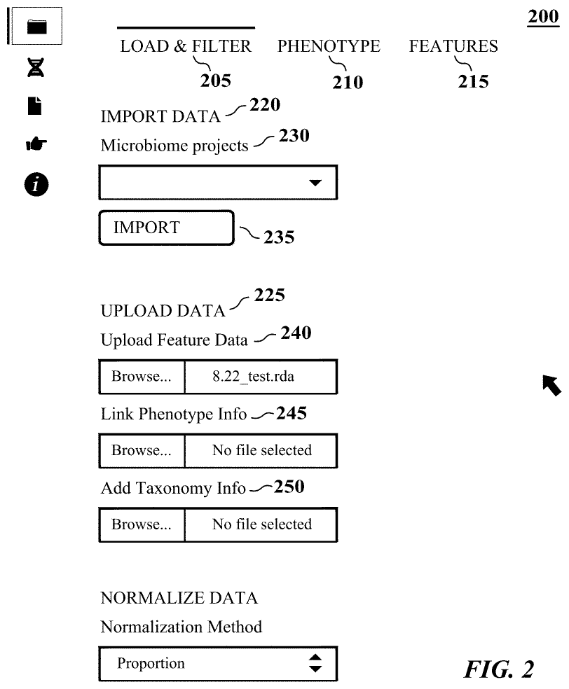

FIG. 2 illustrates an example initial interface 200 of the software tool 120. In particular embodiments, the initial interface 200 may provide a selection of tabs, including tabs for loading and filtering data (load & filter 205), phenotype selection (phenotype 210), feature selection (features 215), etc. With the selection of the “load & filter 205” tab, the user may be navigated to the data input interface, which may provide selections for the user to import data 220, upload data 225 and select different settings. If a microbiome project was previously created with data already uploaded to the software tool 120, the user may import such data by selecting from the “microbiome projects 230” drop-down menu and clicking on the “import” 235 button. Otherwise, the user may upload data externally. As an example and not by way of limitation, the user may upload feature data 240 when uploading data. As another example and not by way of limitation, the user may link phenotype information 245 when uploading data. As yet another example and not by way of limitation, the user may add taxonomy information 250 when uploading data.

In particular embodiments, the software tool 120 may accept several different data/file formats comprising metagenome sequencing results which may be stored at an MRexperiment file (a modified object for the data from high-throughput sequencing experiments) internally. If an MRexperiment file is already available, a user may simply upload it as, e.g., an RData file (designed for use with R) or an RDS object (a single R object). In particular embodiments, the software tool 120 may determine the correct file format based on the file extension. If an MRexperiment file is not already available, biological observation matrix (BIOM) formatted files produced by any program may also be provided. As an example and not by way of limitation, such program may comprise QIIME2 (a computing environment for processing and analyzing amplicon library data) or mothur (an open source software package for bioinformatics data processing). If an MRexperiment file is not already available, a simple counts matrix in CSV (comma-separated values) or TSV (tab-separated values) format may be also uploaded. In particular embodiments, the software tool 120 may use a counts matrix which may have no row names to store the information on the features or operational taxonomic units (OTUs) in its first columns. All other columns names may correspond to sample names. If the data does not contain phenotype information or in case the user wants to adjust the phenotype information included with an MRexperiment or BIOM file, they may upload an additional phenotype file and link it with the counts data. The required format may be such that each sample is a row with the names of the rows in the phenotype data corresponding to the names of the columns in the count data. Appending several phenotype files by subsequent uploads may be possible. If no phenotype file is given, the names of the columns of the count data may be used as the phenotype data. Finally, if not already included with a given MRexperiment, a feature data file may be provided if aggregation to a particular phylogenetic level is desired. In particular embodiments, each unique feature may be a row corresponding to the ones in the counts data feature column and each column may be a taxonomy level. All three data entities (counts, phenotypes and features) may be stored together internally as an MRexperiment, which may be downloaded (if desired) via a “Get Data” option on the data input interface of the microbiome analysis tool.

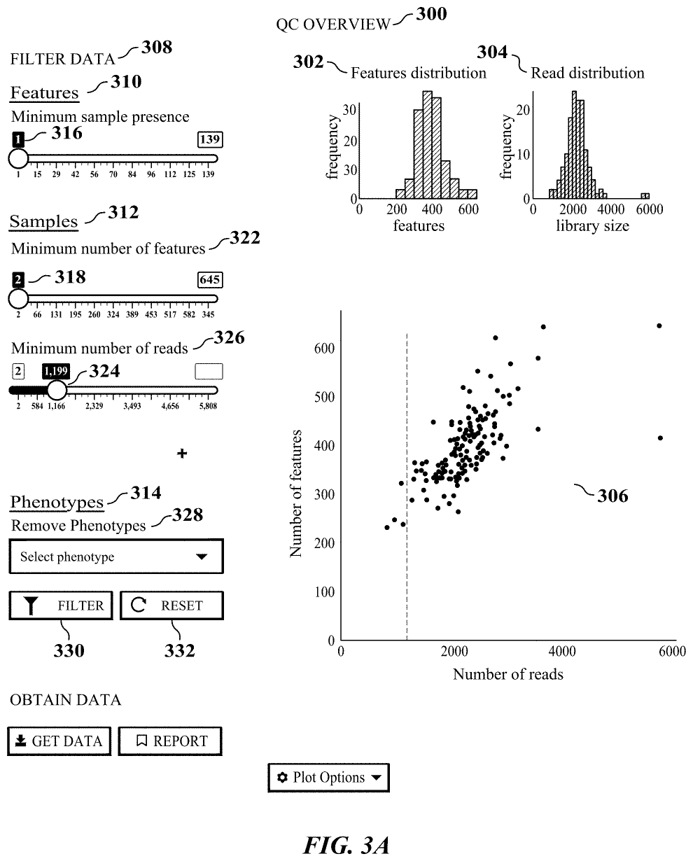

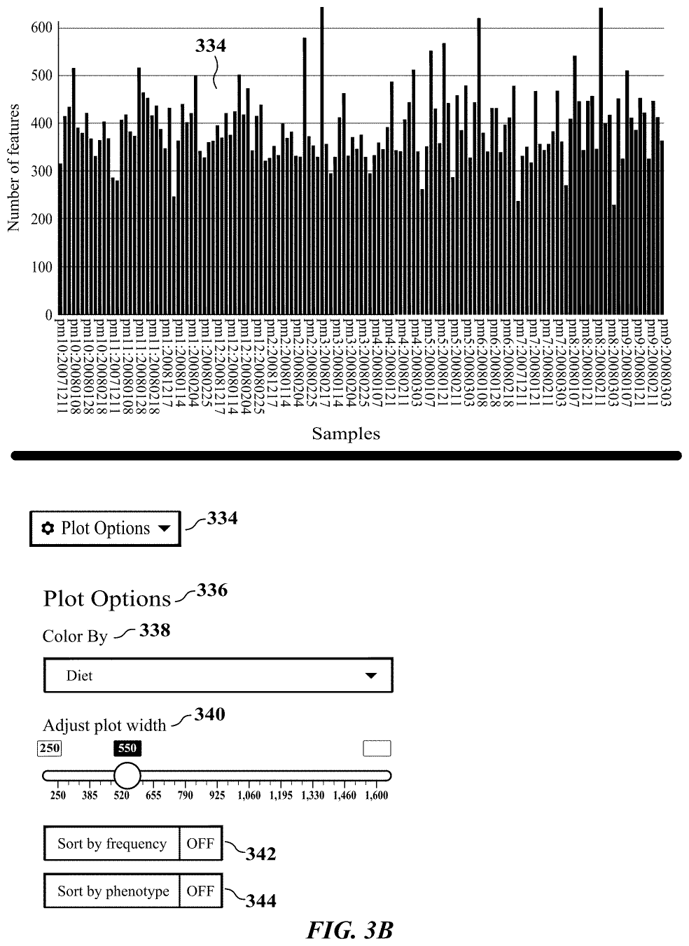

In particular embodiments, the software tool 120 may generate one or more quality-control plots based on the accessed microbiome samples. Once the data has been uploaded, several QC plots may give an overview of the number of features and the number of reads available in each of the samples. FIG. 3A illustrates an example user interface for an overview of quality-control (QC) 300 plots. As an example and not by way of limitation, the QC plots may comprise feature distribution 302, read distribution 304, and sequencing statistics 306 plotting the number of features against the number of reads. The QC plots may provide annotations in the plot to provide additional detail for each plot point. Within this user interface, the software tool 120 may provide functionalities of various methods for normalizing data. In particular embodiments, samples with poor quality may be filtered through exploratory data analysis of the sequencing depth and number of features observed. Normalization may be enabled to calculate proportions or by using cumulative sum scaling (CSS).

In particular embodiments, the software tool 120 may receive, via the user interface of the software tool, a data-filtering input based on one or more of minimum sample presence, minimum number of features, or minimum number of reads. As illustrated in FIG. 3A, the user may filter the inputted samples using different options under the filter data 308 tab. These options may comprise features 310, samples 312, phenotypes 314, etc. As an example and not by way of limitation, the user may adjust a slider 316 requiring minimum sample presence 318. As another example and not by way of limitation, the user may adjust a slider 320 requiring a minimum number of features 322 present in a sample or a slider 324 requiring a minimum number of reads 326 that a feature needs to be present in. Any changes in the slider settings may be immediately shown in the QC plot to allow the user to see the effect such filtering may have. In addition to threshold-based filtering, the user may also subset the samples by removing phenotypes 328 by selecting a specific phenotype. In particular embodiments, any levels that are not of interest may be selected (these may not be reflected visually in the QC plot). The user may proceed with the data filtering by clicking on the “filter” 330 button, which may generate the subset of the data structure accordingly. In other words, the software tool 120 may generate a subset of the accessed microbiome samples based on the data-filtering input. In order to get back to the original dataset, the user may select the reset option, e.g., by clicking on the “reset” 332 button. In particular embodiments, the software tool 120 may further update the one or more quality-control plots based on the subset of the accessed microbiome samples. FIG. 3B illustrates an example user interface for a bar plot. In addition to the basic QC plots showing distribution of number of reads and number of features, a sample-based bar plot 334 may give an overview of the number of base features (e.g., OTUs) available for each sample. The software tool 120 may provide different plot options 336 for users. These options 336 may include coloring phenotypes 338, adjusting plot width 340, sorting by frequency 342, and sorting by phenotype 344.

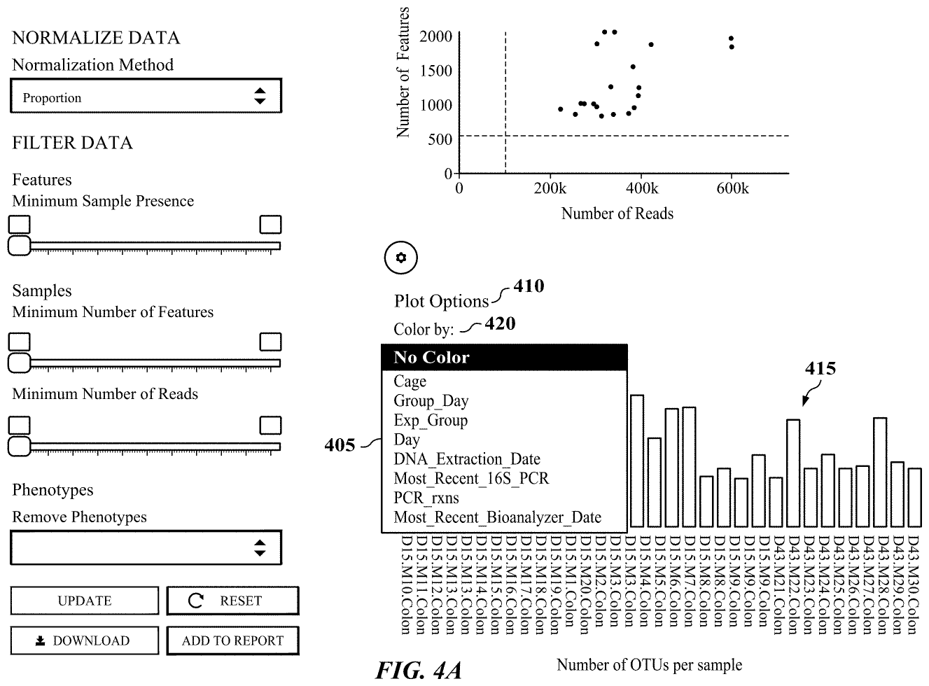

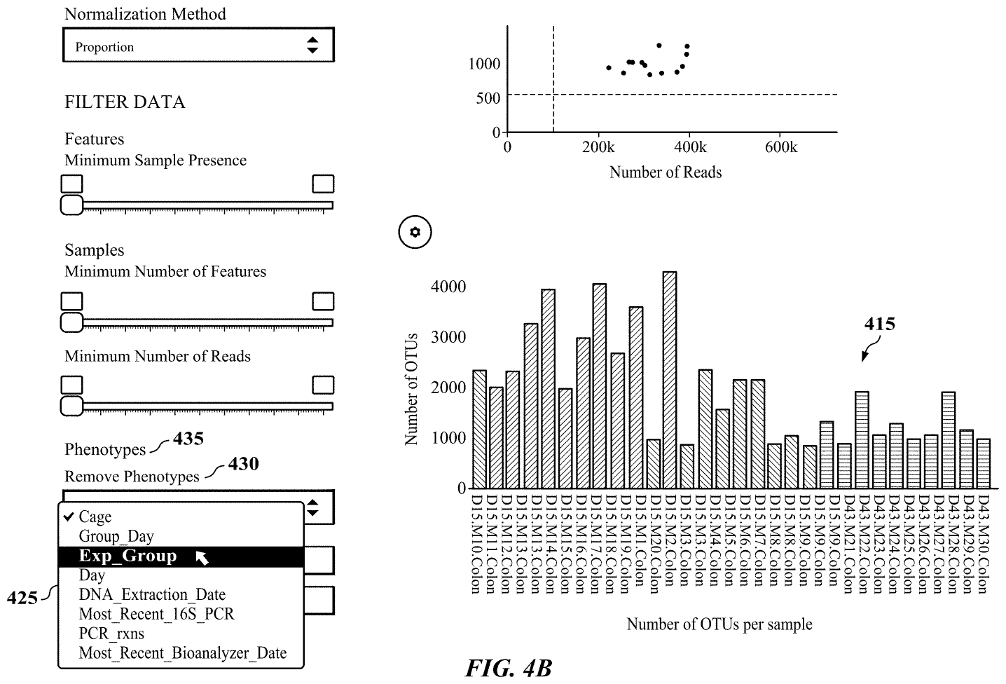

FIG. 4A illustrates an example user interface for a bar plot with color options. As indicated in FIG. 4A, the user may select “no color” from the drop-down menu 405 in plot options 410 for the bar plot 415. As can be seen in FIG. 4A, other example coloring options (“color by” 420) may comprise by cage, by day, by DNA extraction date, by most recent bioanalyzer date, etc. FIG. 4B illustrates an example user interface for a bar plot with phenotype options. As indicated in FIG. 4B, the user may remove certain phenotypes in the drop-down menu 425 of removing phenotype 430 in the input field of phenotypes 435, including those of a control group. As can be seen in FIG. 4B, the user may also remove phenotypes 430 by cage, day, DNA extraction date, most recent bioanalyzer date, etc. All of the plots as aforementioned in FIGS. 3A-3B and FIGS. 4A-4B may be included in the reports if the user selects the “report” option on the user interface, e.g., the “add to report” option.

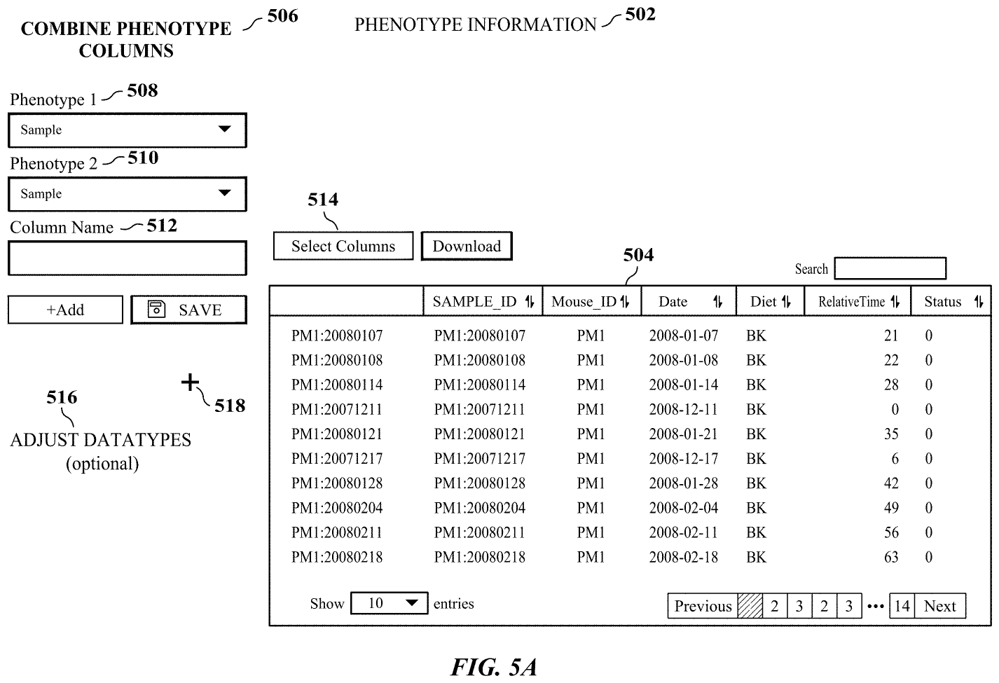

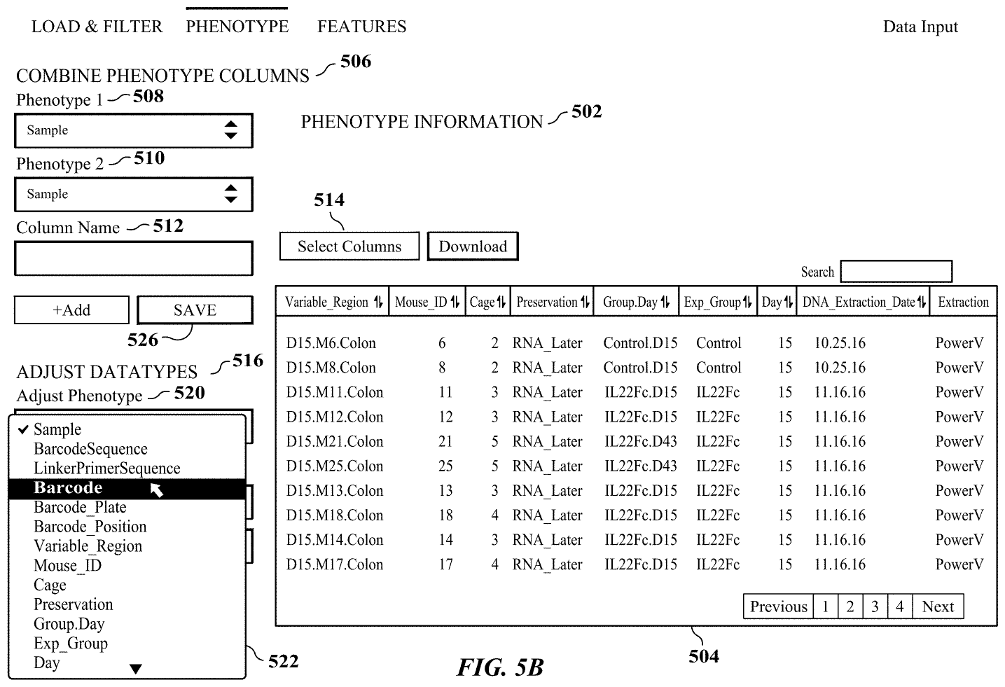

FIG. 5A illustrates an example user interface for phenotype information. In particular embodiments, the user interface for phenotype information may display various data related to or characterizing the phenotypes (e.g., phenotype information 502) in the uploaded data and provide options to select phenotypes and their interactions. As indicated in FIG. 5A, the user interface for phenotype information may show the available phenotype associated with the feature count set in an interactive table 504. The main purpose of this table 504 may be informational, but if desired the user may create new phenotype columns by combining existing ones. As an example and not by way of limitation, the user interface for phenotype information may allow the user to combine phenotype columns 506 by selecting a first phenotype (phenotype 1 508) and a second phenotype (phenotype 2 510). The user may further specify the column name 512 for the newly created phenotype. This may enable the creation of new data to center the analysis around, i.e., the values of the two columns may be pasted together creating an interaction value. The user may furthermore select columns 514 to show in the table 504 and remove those irrelevant for the analysis. If desired, the data type of each column may be modified by opening the adjust datatypes 516 box via clicking on the “+” 518 icon. This may be not required but may be of interest for specific analysis requests. FIG. 5B illustrates an example user interface for adjusting the plot datatype. In particular embodiments, the adjustment of datatypes may enable the software tool to take a vector in the interactive table 504 and encode what the R class for the vector should be, e.g. numeric, character, factor, etc. This feature may be important as certain functions may require a type of class and at times users may unintentionally upload data formatted differently from the requirement. As an example and not by way of limitation, a series of ages for a patient may be uploaded. There may be a typo, e.g., one sample has the text “7” instead of a numeric entry. This typo may render the entire column a character vector, for which distributional analyses may be invalidated, and one may unable to generate a scatterplot of age versus abundance, etc. With the ability to adjust the datatype via the user interface, a user may change “7” to 7 to solve the problem.

As indicated in FIG. 5B, the user may adjust phenotype 520 with different options in the drop-down menu 522 in the input field for adjusting datatypes 516. As an example and not by way of limitation, the drop-down menu options 522 may comprise metadata associated with the sample of interest. After selecting an option from the drop-down menu 522, the user may further select how to change the data type. As an example and not by way of limitation, changing the data type may be based on numeric, character, factor, etc. The phenotype information 502 may be updated accordingly, which may be reflected in the updated content in the interactive table 504. Any modifications made may be only stored in the internal MRexperiment for the analysis after pressing the “save” 526 button. All saved modifications may be automatically included in the reports.

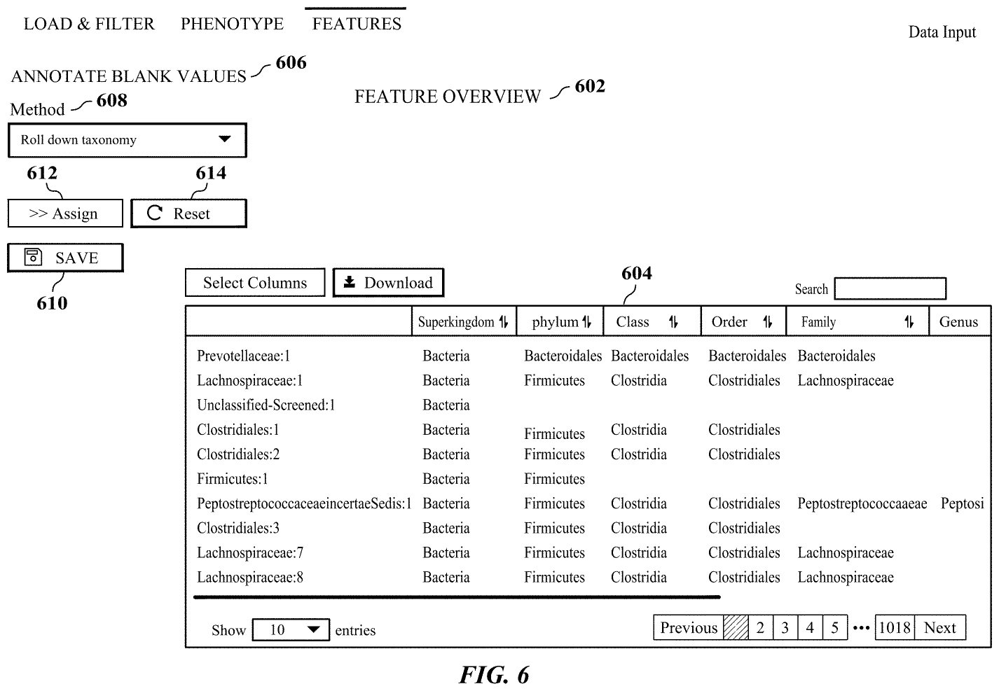

FIG. 6 illustrates an example user interface for an overview of microbiome features. As illustrated in FIG. 6, the user interface for features may provide feature overview 602. As an example and not by way of limitation, the feature overview 602 may comprise available feature taxonomy for the counts data, which may be listed in a feature table 604. In particular embodiments, the user interface for microbiome features may include an annotate option, e.g., “annotate blank values” 606. The user may select different methods 608 for annotation. As an example and not by way of limitation, the method 608 may be rolling down taxonomy or mark as unknown. In particular embodiments, the annotate option may apply a high-level annotation to a group of data, for which more detailed annotation(s) may be unknown or unclear (e.g., low in confidence level). The user interface for microbiome features may also include a save option 610. The save option 610 may ensure that features saved in the feature table 604 displayed at the user interface are used in future visualizations or analyses. As indicated in FIG. 6, the user interface for microbiome features may show the available feature annotation associated with each raw count feature (e.g., OTU). In case of missing values which may occur for features that cannot be annotated down to a specific taxonomy level, the user may have the option to either mark them as unknown (to include “unknown” as an annotation value) or to roll down the taxonomy. In this case, the highest annotated taxonomy level may be reused at more specific taxonomy level with the work “unknown_” prepended. For analysis at a more specific taxonomy level, this may allow summary functions to distinguish between features of higher order taxonomy levels. These changes may be calculated and shown in the table 604 after clicking the “assign” 612 button and may be reverted via the “reset” 614 button. Any modifications made may be only stored in the internal MRexperiment for the analysis after pressing the “save” 610 button. All saved modifications may be automatically included in the reports.

After all required modifications to the counts, phenotype or feature annotation data have been completed, the user may switch to the analysis section of the software tool 120. The analysis workflow within the software tool 120 may be split into six different sections: intra sample 130, inter sample 135, correlation 140, differential abundance 145, longitudinal 150, and survival 155. Each plot within a corresponding analysis section may be generated after the user sets all required input elements and clicks the “update” button. All visualizations may be implemented using the plotly R package which provides basic interactivity, including zooming or panning via its mode bar. In addition, the user may export the plot in its current state (i.e., showing specific user interactions) as a scalable vector graphic (SVG) file using the camera icon of the mode bar. Every analysis section may comprise one or more “report” buttons associated with its respective input fields. In order to include the results of the current analysis, i.e., the currently visible plots, the user may click the “report” button which may include code to reproduce the visualization outside of the software tool.

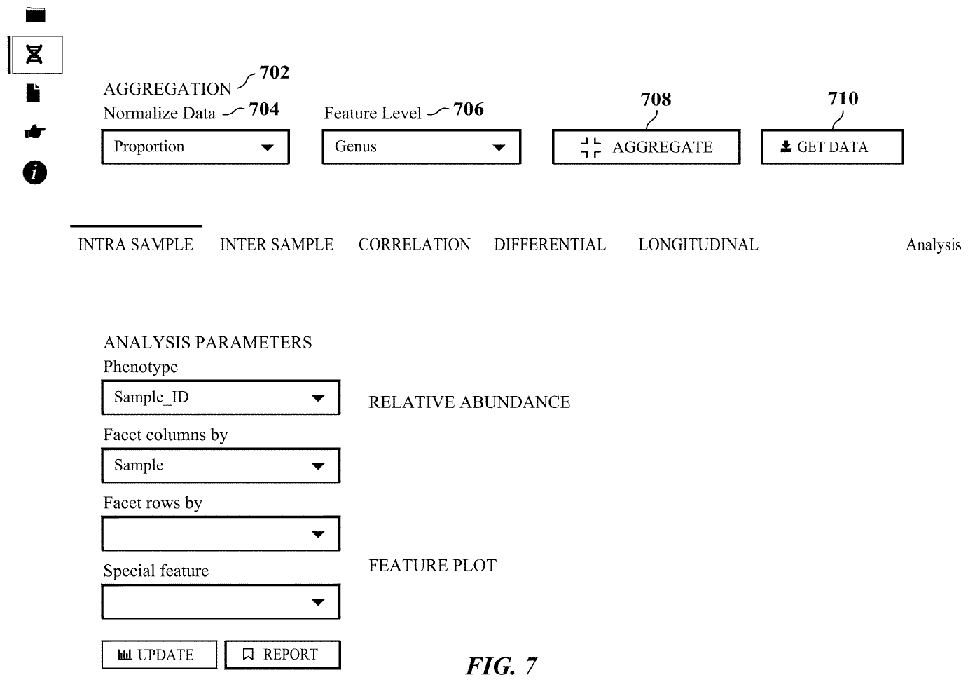

FIG. 7 illustrates an example user interface for data aggregation. Before any analyses can be done, the data may have to be aggregated to a specific taxonomy level. In case of a missing feature annotation, this may be limited to the raw counts data. In those cases, aggregating may not change the underlying counts, but still need to be performed. For all other taxonomy levels, features may be grouped by their annotation and counts may be summed up for each group. Normalization may allow for the user to account for library size differences before analysis and the user may be required to make a choice on normalization methods while aggregating the data. Certain functions of the software tool may be restricted if none is selected (e.g., percentage is unavailable if not normalized). As an example and not by way of limitation, differential abundance testing may also require normalization, which may be performed silently if the user does not choose to do so. In particular embodiments, the software tool 120 may receive, via the user interface of the software tool, a data-aggregation input specifying a feature level. The software tool 120 may then aggregate the accessed microbiome samples to the specified feature level. As indicated in FIG. 7, there may be an aggregation 702 section, where the user may select different ways to normalize data 704 and select a feature level 706 (e.g., genus) for the data be aggregated to. In particular embodiments, the two available methods available for aggregation may be based on either calculating proportions or using cumulative sum scaling (CSS), which may be selected from a drop-down menu of options when normalizing data 704. The user may also select an option not to normalize the data from the drop-down menu 704. The user may further click on the “aggregate” 708 button to perform the aggregation. If desired the user may choose to download the counts data after aggregation by clicking the “get data” 710 button. The aggregation box may automatically close after aggregation is complete and analysis input areas may become enabled. In order to switch to a different aggregation level, the user may open the aggregation section by clicking the “+” icon associated with the box.

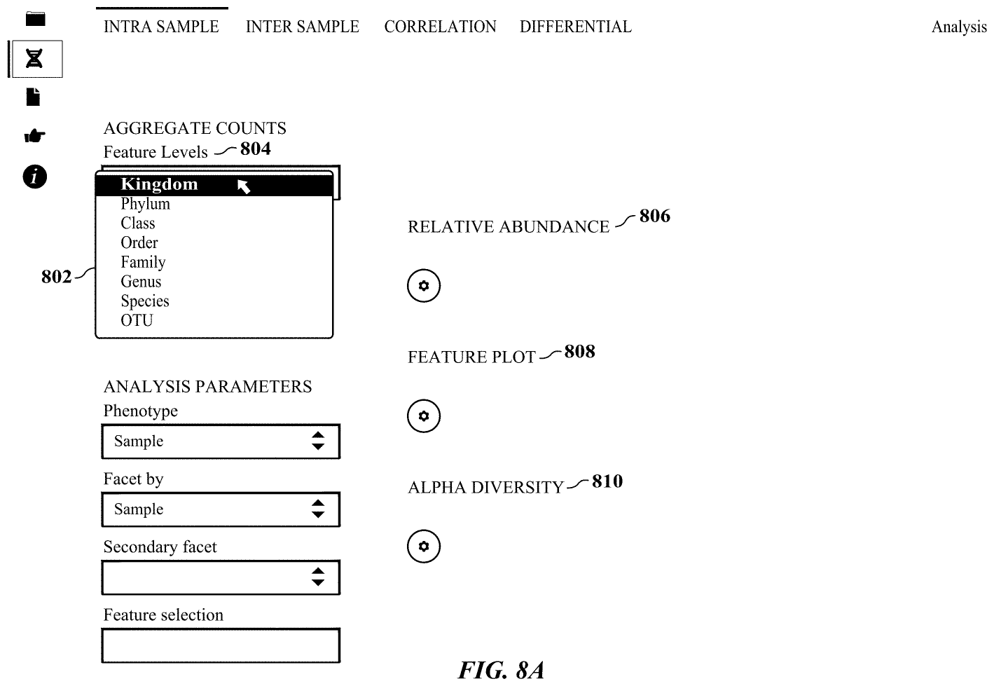

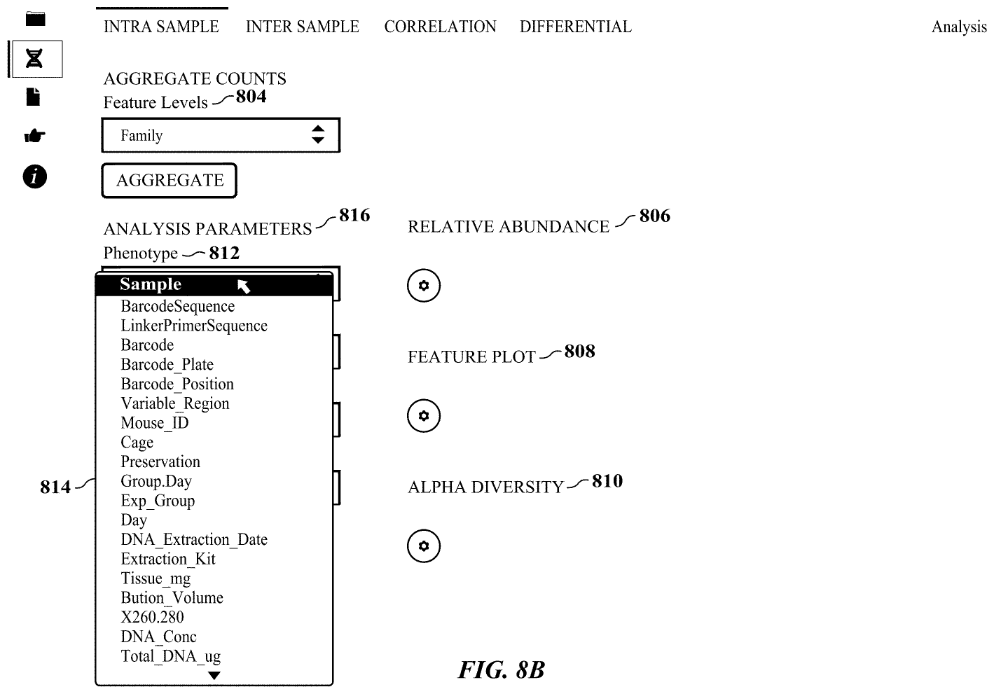

In particular embodiments, the microbiome analyses may comprise an intra-sample analysis 130 comprising investigating microbial composition within a microbiome sample or a group of microbiome samples. The intra-sample analysis 130 may compare compositions in each sample or groups or samples. For intra-sample analysis 130, the one or more analysis results may comprise one or more of relative abundance, feature abundance, or alpha diversity. Different functions may be available to visualize the relative abundance of top features, the abundance of a specific feature as well as the alpha diversity within the sample. Within the software tool 120, one common set of input elements may be used to generate all visualization. FIG. 8A illustrates an example user interface for selecting the feature level for intra-sample analysis. As illustrated in FIG. 8A, the user may select a feature level in the drop-down menu 802 of feature levels 804 for intra-sample analysis. As an example and not by way of limitation, the user may have selected kingdom as the feature level in FIG. 8A. Accordingly, the software tool 120 may show analysis results of relative abundance 806, feature plot 808, and alpha diversity 810 based on the selected feature level 804. FIG. 8B illustrates an example user interface for selecting analysis parameters for intra-sample analysis. As illustrated in FIG. 8B, the user may select a phenotype 812 in the drop-down menu 814 in analysis parameters 816. As an example and not by way of limitation, the user may have selected sample as the phenotype 812 in FIG. 8B. Accordingly, the software tool 120 may show analysis results of relative abundance 806, feature plot 808, and alpha diversity 810 based on the selected phenotype 812. In particular embodiments, the software tool 120 may show relative abundance 806, feature plot 808, and alpha diversity 810 upon receiving user inputs with respect to feature levels 804 and analysis parameters 816. As an example and not by way of limitation, the user may first select family as the feature level 804 and then select sample as phenotype 812 as indicated in FIG. 8B to get the relative abundance 806.

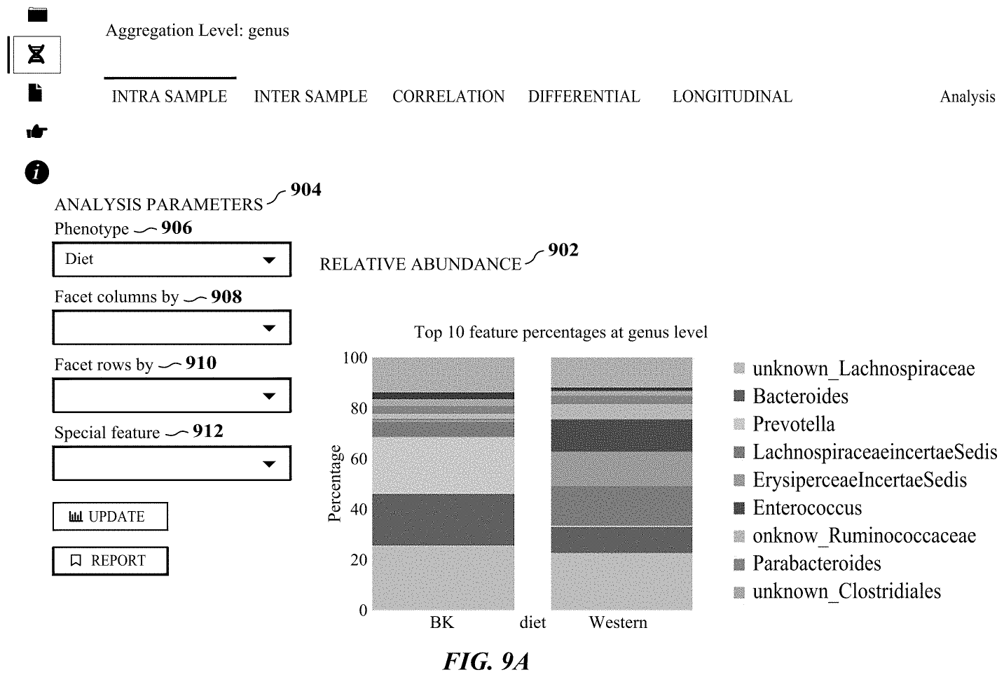

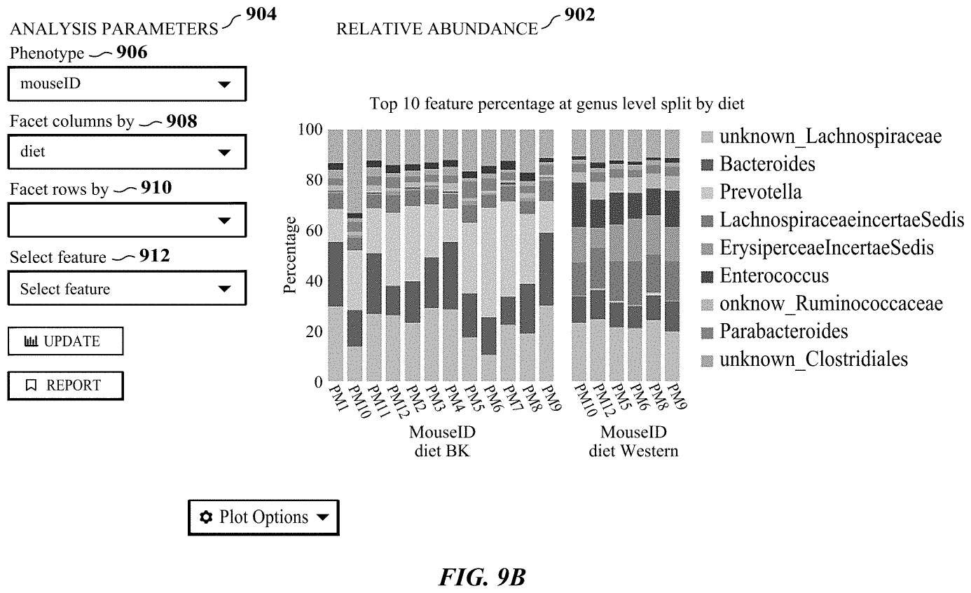

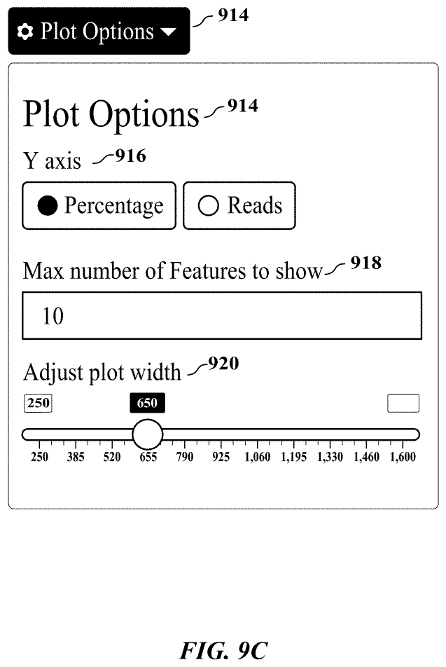

In particular embodiments, the one or more analysis results may comprise relative abundance. The relative abundance may comprise one or more abundant features in a bar plot generated based on one or more user-specified analysis parameters. In particular embodiments, the one or more user-specified analysis parameters may comprise one or more of a phenotype, a faceting manner, or a feature. The bar plot may be modifiable based on one or more of a number of features to show, a switch between showing percentage or reads, or a plot width. As an example and not by way of limitation, relative abundance may show the most abundant features in a bar plot summarized by a user-defined variable across the x-axis. In addition, the user may choose to facet by phenotypes, adjust the number of features to show, switch between showing total numbers (reads) and normalized value (if normalized), and modify the overall plot width. In general, main input elements may be defined before any visualization can be produced whereas the plot options may only modify existing plots if deemed necessary. FIG. 9A illustrates an example user interface for relative abundance. As indicated in FIG. 9A, the relative abundance 902 may show the top 10 feature percentage at genus level. Besides genus level, counts may be aggregated to other feature levels including kingdom, phylum, class, order, family, specifies, and OTU. The user may select different analysis parameters 904, which may affect the visualization of relative abundance 902. In particular embodiments, the analysis parameters may comprise one or more of phenotype 906, a way of faceting columns (facet columns by 908), a way of faceting rows (facet rows by 910), or a selected feature (select feature 912). As an example and not by way of limitation, phenotype may comprise sample, barcode sequence, linker primer sequence, barcode, barcode plate, barcode position, variable region, mouseID, cage, preservation, group.day, control group, day, DNA extraction date, extraction kit, tissue (mg), elution volume, etc. As another example and not by way of limitation, the way of faceting columns/rows may comprise faceting by cage, group.day, control group, day, DNC extraction date, most recent 16 S PCR, PRC_rxns, or most recent bioanalyzer date. As another example and not by way of limitation, the options for selecting feature 912 may comprise taxonomic information, such as unknown Lachnospiraceae, Bacteroides, Prevotella, Lachnospiraceae Incertae Sedis, Erysipelotrichaceae Incertae Sedis, Enterococcus, unknown Ruminococcaceae, etc. As illustrated in FIG. 9A, the user may have selected diet as the phenotype 906, which may be reflected by the relative abundance 902. FIG. 9B illustrates an example user interface for relative abundance with facet options. As indicated in FIG. 9B, the user may select by which the columns may be faceted. As indicated in FIG. 9B, the phenotype 906 for the relative abundance 902 may be mouseID whereas the columns may be faceted by diet. Accordingly, the relative abundance 902 may show the top 10 feature percentage at genus level split by diet. FIG. 9C illustrates an example user interface for selecting plot options for relative abundance. As indicated in FIG. 9C, the plot options 914 may allow the user to select to plot the relative abundance either by percentage or by reads for Y axis 916. In addition, the user may specify the maximum number of features to show 918 and adjust the plot width 920. As an example and not by way of limitation, FIG. 9C illustrates that the user specified 10 as the maximum number of features to show and 650 as the plot width. With a click on a specific feature in the plot, the software tool may automatically open a feature abundance plot for this feature.

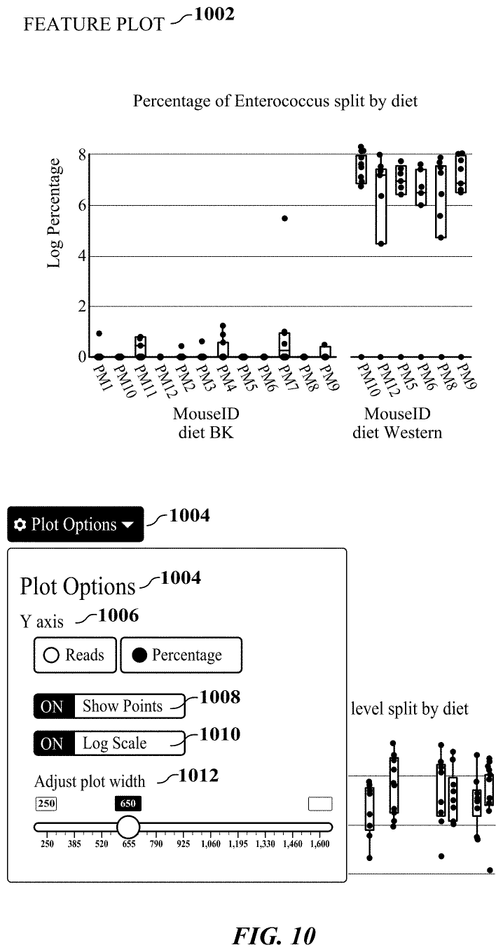

In particular embodiments, the one or more analysis results may comprise feature abundance. The feature abundance may comprise an individual abundance of a specific feature as a box plot or a categorical scatterplot generated based on one or more user-specified analysis parameters. The one or more user-specified analysis parameters may comprise one or more of a phenotype, a faceting manner, or a feature. In particular embodiments, the box plot or categorical scatterplot may be modifiable based on one or more of a switch between showing points or not showing points, a switch between showing log scale or not showing log scale, a switch between showing percentage or reads, or a plot width. In particular embodiments, the feature abundance plot may show the individual abundance of a specific feature either as a boxplot or a categorical scatterplot depending on the x-axis variable chosen. The user may choose to employ a particular scale, define plot width and decide whether to show individual sample points or not. Feature plots may be opened by selecting a specific feature in the input section or by clicking on a feature in the relative abundance plot. FIG. 10 illustrates an example user interface for feature abundance with plot options. As illustrated in FIG. 10, the feature abundance plot 1002 may show the percentage of enterococcus split by diet. The plot 1002 may additionally show the mouseID as diet BK and the mouseID as diet Western. The user interface may further show plot options 1004 that the user may select. As an example and not by way of limitation, the user may select reads or percentage for Y axis 1006. As another example and not by way of limitation, the user may determine whether to show points 1008 or log scale 1010 with a corresponding on/off switch button (e.g., “on” for both options as illustrated in FIG. 10). As yet another example and not by way of limitation, the user may adjust the plot width 1012 via a slider (e.g., 650 as illustrated in FIG. 10).

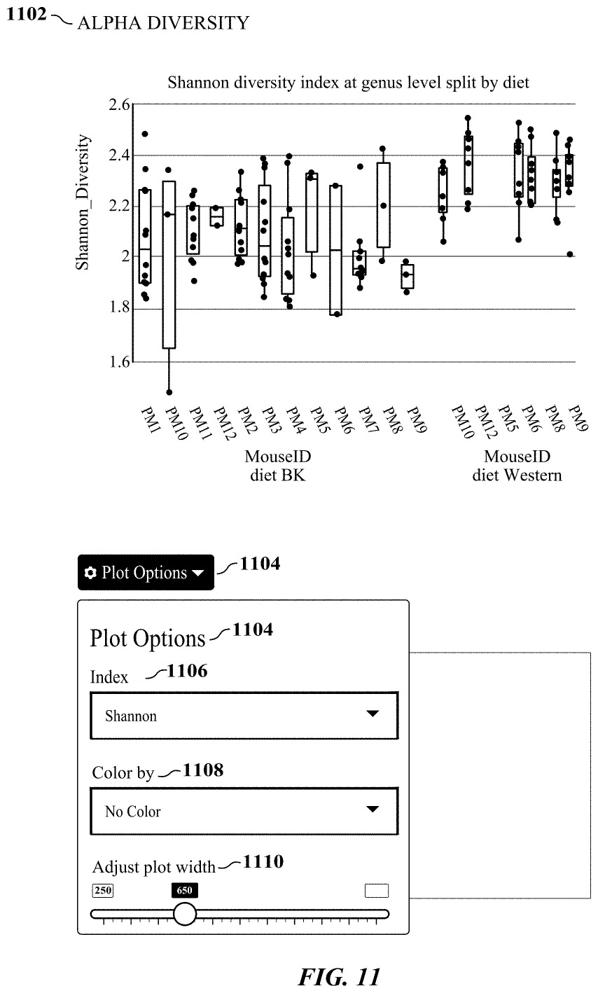

In particular embodiments, the one or more analysis results may comprise alpha diversity. The alpha diversity may comprise a measure of a complexity or a diversity within a particular microbiome sample (e.g., habitat or area) as a box plot generated based on one or more user-specified analysis parameters. In particular embodiments, the one or more user-specified analysis parameters may comprise one or more of a phenotype, a faceting manner, or a feature. The box plot may be modifiable based on one or more of an index, a coloring manner, or a plot width. Alpha diversity may be computed by functions in the vegan package and may be visualized as a boxplot using the same input definitions by feature and relative abundance. The user may choose to color and thus split the boxes by a phenotype and set the overall plot width. In particular embodiments, multiple diversity measures may be offered with Shannon diversity provided as the default. Shannon diversity in particular may measure how evenly the microbes are distributed in a sample and may be defined based on the proportion of an individual feature. The diversity measure may be changed via the plot options and the plots may be split by a specific phenotype coloring. FIG. 11 illustrates an example user interface for alpha diversity with plot options. As illustrated in FIG. 11, the alpha diversity plot 1102 may show the Shannon diversity index at genus level split by diet. The plot 1102 may additionally show the mouseID as diet BK and the mouseID as diet Western. The user interface may further show plot options 1104 that the user may select. As an example and not by way of limitation, the user may select different index 1106 besides Shannon, including Simpson, inverse Simpson, richness, etc. As another example and not by way of limitation, the “color by” 1108 option may allow the user to select coloring the plot based on different factors (e.g., no color as indicated in FIG. 11). As yet another example and not by way of limitation, the user may adjust the plot width 1110.

In particular embodiments, the microbiome analyses may comprise an inter-sample analysis 135. The inter-sample analysis 135 may comprise determining differences between microbiome samples or a group of microbiome samples. The inter-sample analysis 135 may focus on differences between samples or groups of samples via feature heatmaps and beta diversity calculations. Accordingly, the one or more analysis results may comprise one or more of beta diversity or feature heatmap. The inter-sample analysis 135 may enable a user to analyze microbiome differentiations between samples or phenotype groups. Based on inter-sample analysis 135, the software tool 120 may provide beta diversity visualizations and heatmaps. In particular embodiments, responsive to different user selections of the provided options, the software tool 20 may show the extent to which a given variable is contributing to beta diversity or heatmap coloring. The software tool 120 may also display clusters or data of interest via the user interface to a user.

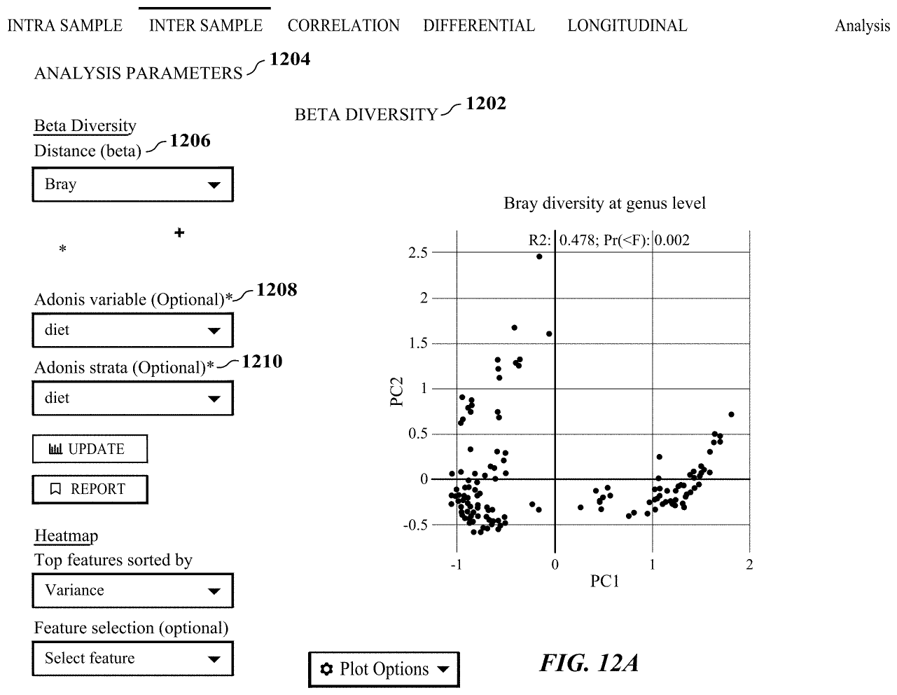

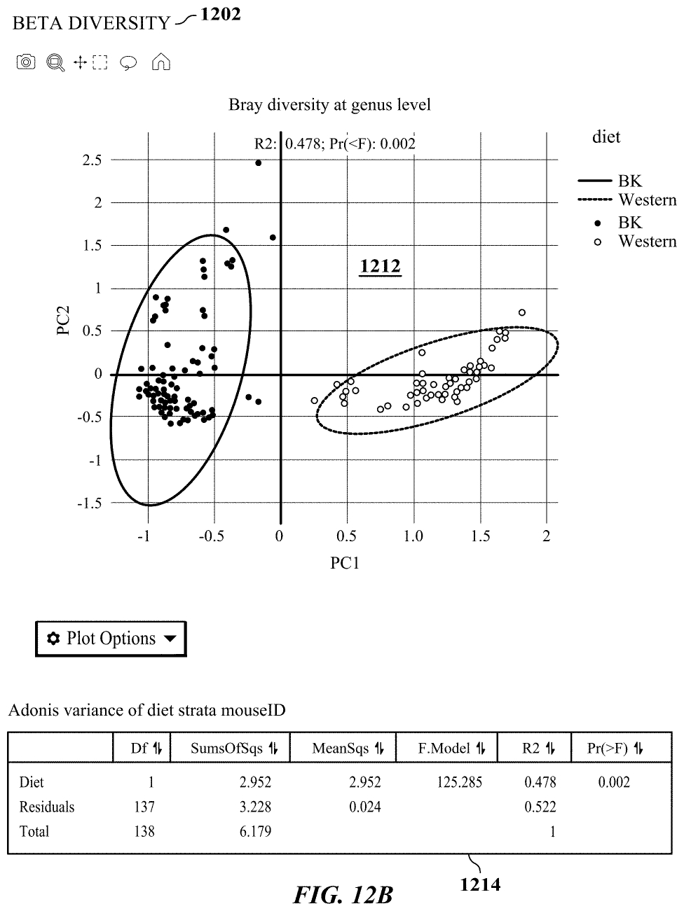

In particular embodiments, the one or more analysis results may comprise beta diversity. The beta diversity may comprise a measure of a complexity of communities between microbiome samples, as compared to within a sample (i.e., alpha diversity). The measure may be based on one or more user-specified analysis parameters. In particular embodiments, the one or more user-specified analysis parameters may comprise one or more of a distance matrix, an Adonis variable, or an Adonis strata. The beta diversity may be illustrated as a scatter plot generated based on principle component analysis. The scatter plot may be modifiable based on one or more of a selection of one or more principal components, a coloring ellipse based on a phenotype, a shape based on a phenotype, a point size, or a plot width. Calculating beta diversity may first require the computation of a pairwise distance or similarity matrix. With the software tool, the user may select between different measures offered via the vegan package. As an example and not by way of limitation, bray may be the suggested default selection of the measures for microbiome analysis. FIG. 12A illustrates an example user interface for beta diversity. As indicated in FIG. 12A, the beta diversity 1202 may be at genus level. The beta diversity 1202 may be generated based on the analysis parameters 1204, as selected by the user. In particular embodiments, the analysis parameters 1204 may comprise one or more of distance 1206, Adonis variable 1208, or Adonis strata 1210. The currently selected distance 1206 by the user may be Bray, Adonis variable 1208 may be diet, Adonis strata 1210 may be mouseID. Other example distance 1206 may comprise distance metrics, such as Canberra, Jaccard, Euclidean, Manhattan, Clark, Kulczynski, Gower, altGower, Morisita, Horn, Mountford, Raup, Binomial, Chao, Cao, Mahalanobis, etc. Other example Adonis variable 1208 or Adonis strata 1210 may comprise sample, cage, control group, day, DNA extraction date, tissue (mg), total DNC, most recent 16 S PCR, most recent bioanalyzer date, etc. Additionally, the top features may be sorted by variance as selected by the user.

In particular embodiments, principal component analysis, a dimension reduction method, may be subsequently performed on the chosen distance matrix and visualized in a scatter plot. The user may have the option to choose the principal components to display, add coloring and confidence ellipses based on a phenotype, define the shape based on a phenotype, and adjust both the point size as well as the overall plot width. In particular embodiments, permutational multivariate analysis of variance (PERMANOVA), from the vegan package may be offered via the software tool 120. In particular embodiments, a user may choose to use command lines to run this function independently and pass the results to the plotting function. A PERMANOVA analysis may let the user statistically determine if the centroids of a dissimilarity or distance matrix differ between groups of samples. Optionally, the user may select a phenotype as well as a strata variable with the results being shown, both within the visualization as well as in a table below it. FIG. 12B illustrates an example user interface for beta diversity with principle component analysis and permutational multivariate analysis of variance. As indicated in FIG. 12B, the software tool 120 may enable the user to perform principle component analysis. The beta diversity 1202 after principle component analysis may be shown in FIG. 12B, as the first principle component (PC1) and the second principle component (PC2), respectively, which may comprise a plot 1212 Bray diversity at genus level. FIG. 12B further illustrates the Adonis variance 1208 of diet with strata mouseID in the table 1214 below the Bray diversity plot.

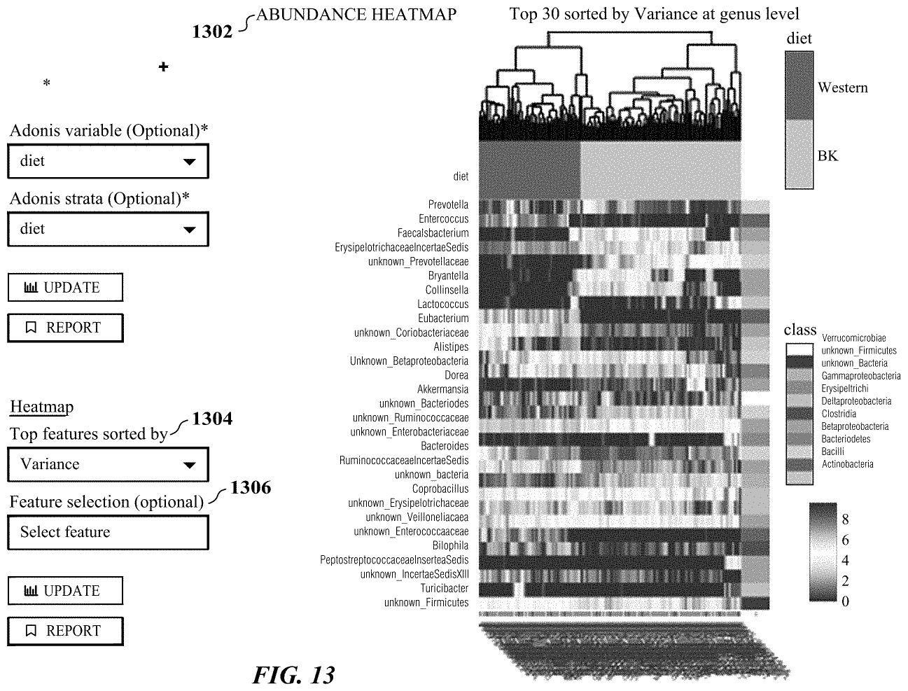

In particular embodiments, the one or more analysis results may comprise feature heatmap. The feature heatmap may comprise a visualization on differences and similarities between microbiome samples. In particular embodiments, the feature heatmap may be generated based on one or more of a number of top features sorted by a user-defined criteria or a user selected feature. The user-defined criteria may comprise one or more of variance, Fano factor, or median absolute deviation. FIG. 13 illustrates an example user interface for abundance heatmap. As illustrated in FIG. 13, the abundance heatmap 1302 may show the top 30 features sorted by variance at genus level. In particular embodiments, the heatmap 1302 may offer another view on differences and similarities between the samples in a dataset. The user may either choose specific features (e.g., using the “feature selection” 1304 option) or show a number of (e.g., 50) top features sorted by different factors (e.g., using the “top features sorted by” 1306 option. As an example and not by way of limitation, these factors used to sort the top features may comprise variance, Fano factor, or median absolute deviation (MAD). In particular embodiments, the visualization may be done with heatmaply which in turns relies on plotly to render the heatmap 1302. The same options to interact with the plot may be thus available. Once rendered, the user may change the number of features to include, turn of log scale, and add annotations to both rows (phenotypes) and columns (higher taxonomy levels) of the heatmap 1302 via the plot options.

In particular embodiments, the microbiome analyses may comprise a correlation analysis 140. The correlation analysis 140 may comprise a visualization of relationship between two features or a feature and a phenotype in a scatter plot enhanced with a linear regression statistic. Faceting and/or coloring by phenotypes may be available in both correlation plots. In particular embodiments, the correlation analysis 140 may be generated based on an association-evaluation method comprising one or more of Spearman, Person, or Kendall. In particular embodiments, Spearman may be the default method. The user may be asked to choose between different methods to aid in the evaluation of the association. The correlation analysis 140 may be further generated based on one or more user-specified analysis parameters comprising one or more of a base feature, a correlation feature, or a correlation phenotype. In particular embodiments, the most common feature may be selected by default as the base feature and the user may have to set the correlation feature or phenotype and may opt to change the base feature. A regression line may be added by default but may be removed via the plot options by the user.

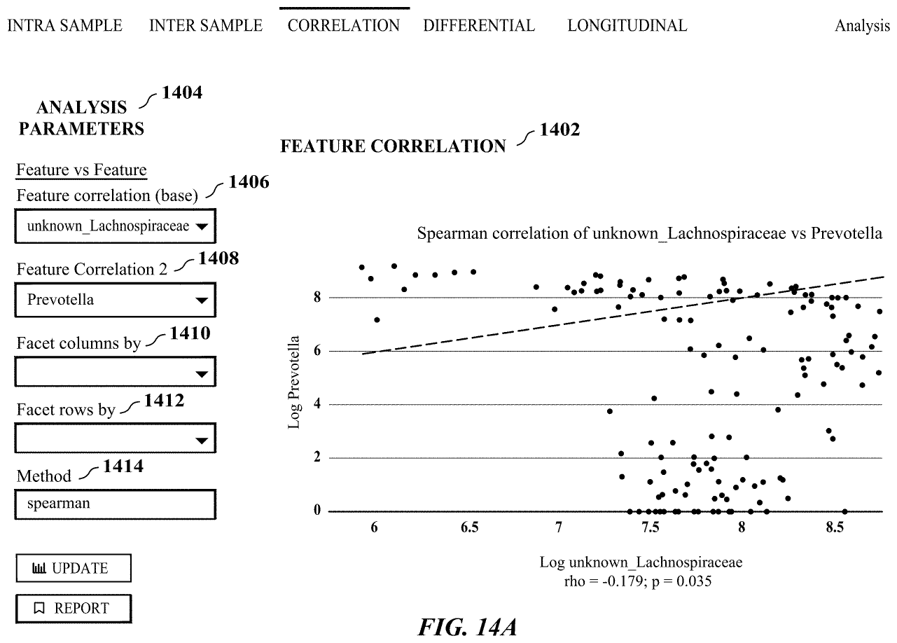

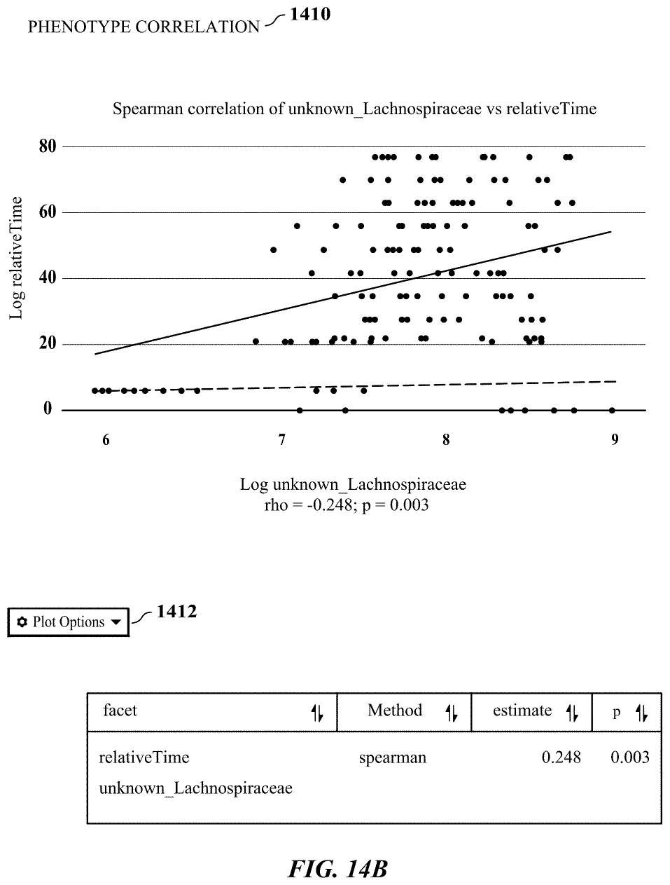

FIG. 14A illustrates an example user interface for feature correlation. In FIG. 14A, the feature correlation 1402 may be the Spearman correlation of two features, i.e., unknown_Lachnospiraceae and Prevotella. The user may select different analysis parameters 1404 with different options provided by the software tool 120. In particular embodiments, the analysis parameters 1404 may comprise one or more of a feature correlation (base) 1406, a feature correlation 2 1408, a manner of faceting columns 1410, a manner of faceting rows 1412, or a method 1414. As an example and not by way of limitation, the user may select “unknown_Lachnospiraceae” as the feature for feature correlation (base) 1406 and “Prevotella” as the feature for feature correlation 2 1408. Other example features for feature correlation (base) 1406 or feature correlation 2 1408 may comprise S24-7, Lachnospiraceae, unknown_o_Clostridiales, Ruminococcaceae, Erysipelotrichaceae, Clostridiaceae, Lactobacillaceae, etc. The user may additionally select by which to facet columns and rows. As an example and not by way of limitation, the facet may be by cage, control group, day, group and day, DNA extraction date, most recent 16 S PCR, most recent bioanalyzer date, etc. The user may further select which method to use to analyze the correlation. Besides Spearman as illustrated in FIG. 14A, the user may select Person or Kendall.

FIG. 14B illustrates an example user interface for phenotype correlation with plot options. In FIG. 14B, the phenotype correlation 1410 may be the Spearman correlation of a feature (unknown_Lachnospiraceae) and a numeric phenotype (relativeTime). The plot options 1412 may enable the user to select by which to facet and which method to use to analyze the correlation.

In particular embodiments, the microbiome analyses may comprise a differential abundance analysis 145. The differential abundance analysis 145 may comprise a test of null hypothesis that a mean or mean ranks between groups of microbiome samples are the same for a specific feature. In particular embodiments, the differential abundance analysis 145 may be generated based on one or more user-specified analysis parameters comprising one or more of a testing method, a comparison phenotype, or a comparison level. The software tool 120 may further generate an interactive table for the differential abundance analysis 145, the interactive table being operable for a user to open feature plots showing specific levels selected by the user. Differential abundance analysis 145 may help detect changes in feature abundance across two or more different levels of a phenotype. In particular embodiments, four different methods may be chosen via the software tool 120, namely DESeq2, Kruskal-Wallis, limma, or a zero-inflated log normal model. DESeq2 and limma are widely used methods for comparisons in microarray and RNA-sequencing data which may easily be adapted for microbiome data. Kruskal-Wallis is a non-parametric test for any differences in distribution between groups. The zero-inflated log normal model may be implemented in the metagenomeSeq package to account for zero-inflation in microbiome data. In particular embodiments, DESeq2 may be used with small (e.g., <=25) sample sizes. The results may be displayed in an interactive table (DT) within the software tool 120 and the user may open feature plots showing the specific levels by clicking on a row of interest.

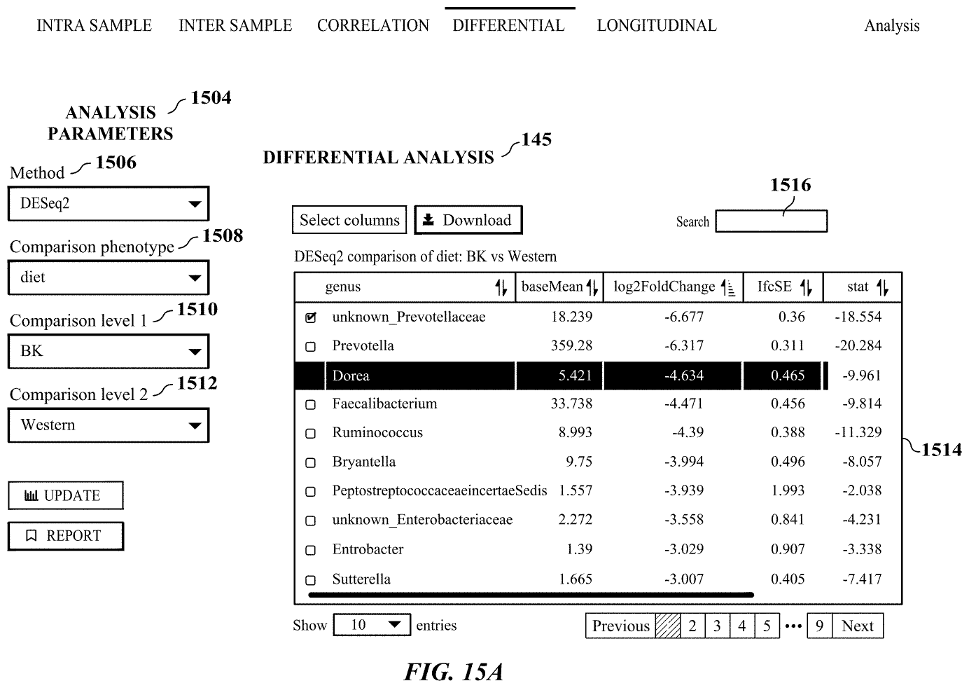

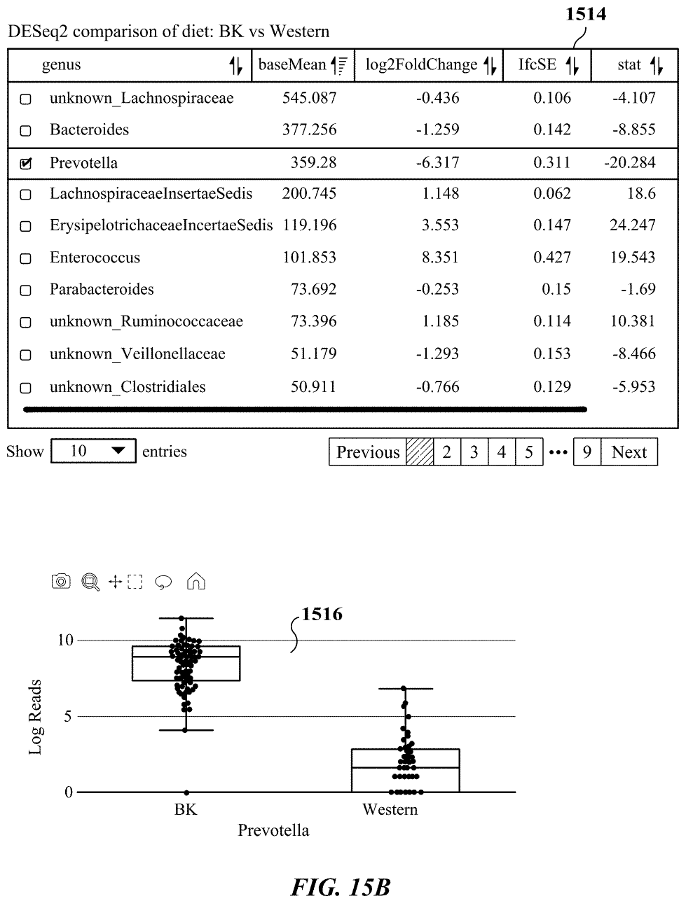

FIG. 15A illustrates an example user interface for differential analysis. In FIG. 15A, the differential analysis 145 may be generated based on analysis parameters 1504 as selected by the user. In particular embodiments, the analysis parameters 1504 may comprise one or more of a method 1506, a comparison phenotype 1508, a comparison level 1 1510, or a comparison level 2 1512. As an example and not by way of limitation, the user may have selected DEseq2 as the method 1506 with other methods 1506 comprising Kruskal-Wallis, limma, or a zero-inflated log normal model, diet as the comparison phenotype 1508, BK as the comparison level 1 1510, and western as the comparison level 2 1512 in FIG. 15A. Other example comparison level for comparison level 1 1510 or comparison level 2 1512 may comprise metadata pertaining to the sample(s) of interest. Therefore, the differential analysis 145 shown in FIG. 15A may comprise a DEseq2 comparison of diet: BK vs Western. The differential analysis 145 may be shown in the interactive table 1514, in which the user may select each row of interest to see the feature plots for the corresponding level. The user may also search for row of interest to quickly navigate to that row via the search box 1516. As an example and not by way of limitation, the Dorea level may be selected to see the corresponding feature plots. The user may also select different methods, comparison phenotype, comparison level 1, and comparison level 2 from the analysis parameters to see varying differential analysis responsive to the changes of these parameters.

FIG. 15B illustrates an example user interface for differential analysis 145 with a specific level. In FIG. 15B, the differential analysis 145 may comprise a DEseq2 comparison of diet: BK vs Western. The differential analysis 145 may be shown in the interactive table 1514, in which the user may select each row of interest to see the feature plots for the corresponding level. As an example and not by way of limitation, the user may have selected Prevotella as indicated in FIG. 15B. Therefore, the corresponding feature plots 1516 may be displayed below the interactive table 1514.

In particular embodiments, the microbiome analyses may comprise a longitudinal analysis 150. The longitudinal analysis 150 may comprise a comparison of microbial composition across time points or conditions (e.g., tissues). In particular embodiments, the longitudinal analysis 150 may be generated based on one or more user-specified analysis parameters comprising one or more of a selected feature, a longitudinal phenotype, a phenotype level order, or a phenotype identifier. The software tool 120 may further generate an interactive visualization of a feature plot corresponding to the longitudinal analysis 150, the interactive visualization being operable for a user to select and color one or more specific phenotype identifiers within the feature plot. The order of the phenotype levels may be preserved in the visualization. The longitudinal analysis 150 may allow the user to generate feature plots with more control over the data shown within the plot. For a specific feature, the user may choose a phenotype and specific levels of that phenotype to show in the plot. The chosen order of the levels may be kept within the visualization which allows sorting by specific dates or tissues among other things. In particular embodiments, all of these input elements may be required to produce a visualization. In addition, if desired and available, the user may choose a specific phenotype to summarize on which will then be connected by lines across the different levels. The user may use the optional phenotype identifier (ID) parameter to add connections between IDs over all selected time points/conditions. The resulting visualization may be interactive and the user may then select and color specific IDs within the plot by clicking on the lines or selecting them via the input element above the plot. In particular embodiments, several different IDs may be selected using different colors via the color select element next to the input element. In order to do this via mouse clicks on the plot, the user may hold the shift key when adding additional line selections. The user may also double click near the edge of the plot to remove selections.

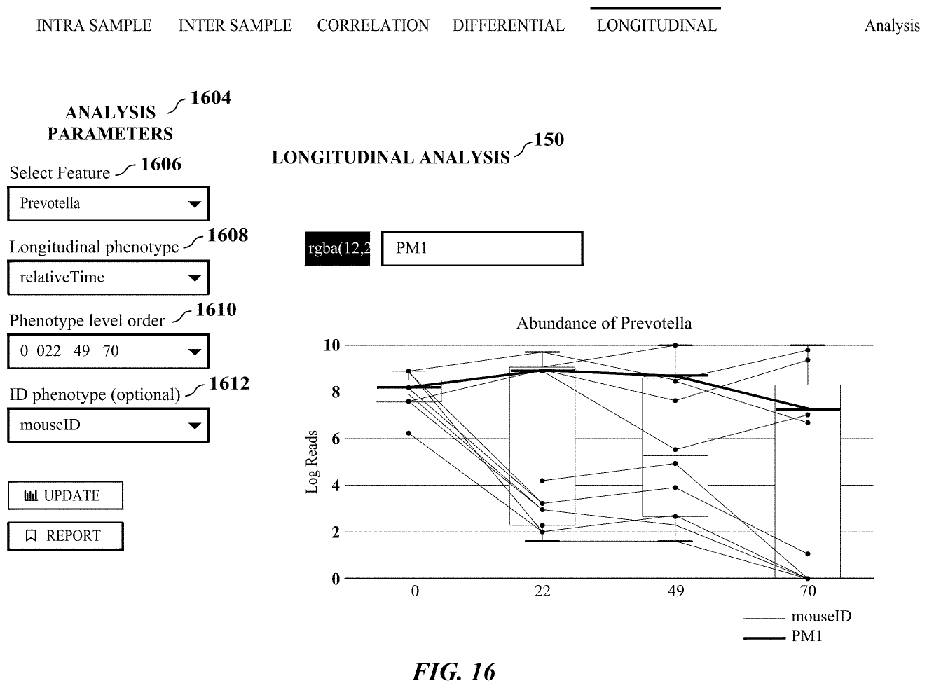

FIG. 16 illustrates an example user interface for longitudinal analysis. In FIG. 16, the longitudinal analysis 150 may be generated based on analysis parameters 1604 as selected by the user. In particular embodiments, the analysis parameters 1606 may comprise one or more of a selected feature 1606, a longitudinal phenotype 1608, a phenotype level order 1610, or a phenotype ID 1612. As illustrated in FIG. 16, the longitudinal analysis 150 may be based on abundance of Prevotella, for which the user may have select Prevotella as the feature 1606, relative time as the longitudinal phenotype, 0, 22, 49, and 70 as the phenotype level order 1610, and mouseID as the phenotype ID 1612. Other example longitudinal phenotype 1608 may comprise phenotypic information relating to the patient or to the sample. Other example phenotype ID 1612 may comprise phenotypic information relating to the patient or to the sample.

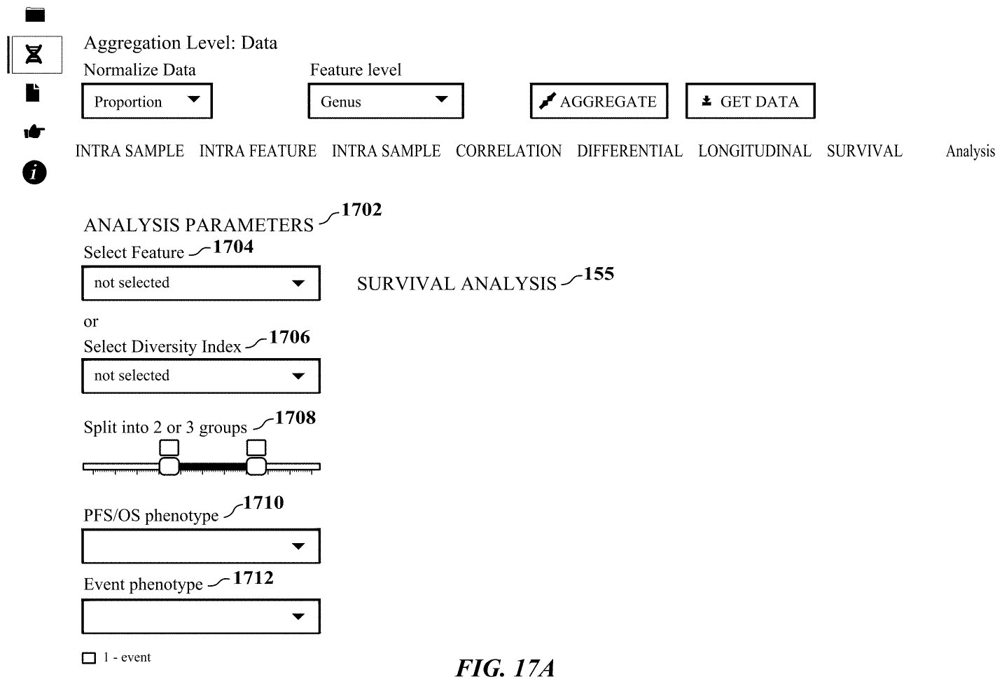

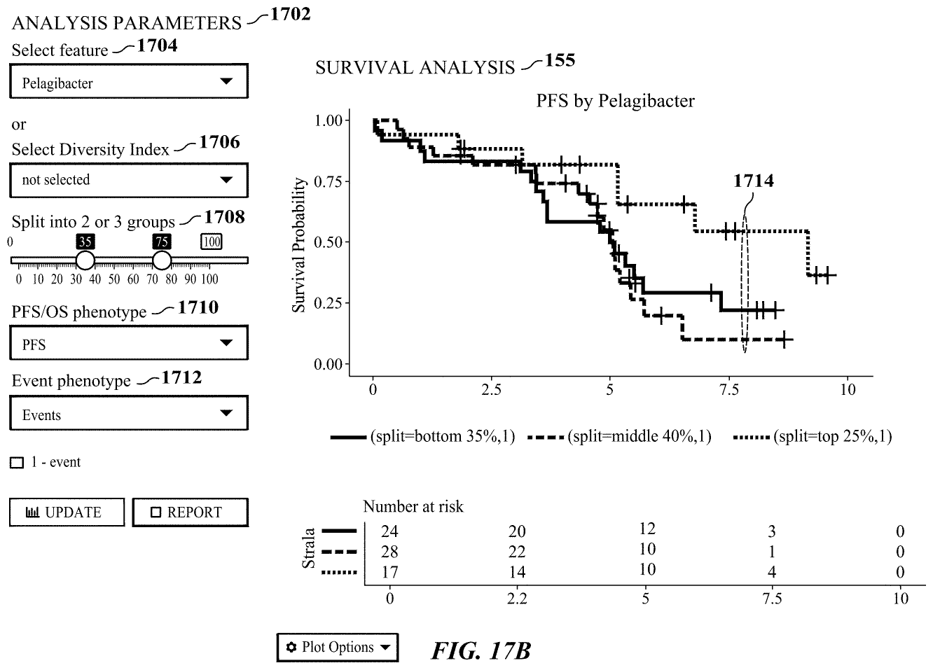

In particular embodiments, the computing system may access a plurality of microbiome samples. The computing system may then receive one or more user-specified analysis parameters associated with a request for a survival analysis 155. The one or more user-specified analysis parameters may comprise one or more of a selected feature, a selected diversity index, a split of groups, or a phenotype. In particular embodiments, the computing system may generate a visualization comprising a result of the survival analysis 155 associated with the plurality of microbiome samples. The result of the survival analysis 155 may be generated based on the one or more user-specified analysis parameters. The computing system may further generate an exportable report and software code comprising the generated visualization. In particular embodiments, a survival analysis 155 may focus on the time to a particular event. As an example and not by way of limitation, the event may be a death if it is about a time to overall survival. As another example and not by way of limitation, the event may be a well-defined progression event. The survival analysis 155 may be based on the time to the event and the censoring, i.e., whether the sample was censored or whether there was a particular event. In particular embodiments, samples being censored may mean they did not die, but it is unknown or we lost them since the time of randomization beyond this particular time, and they have not had a death. If they had an event, that may mean they died at this particular time since randomization. FIG. 17A illustrates an example user interface for survival analysis. As indicated in FIG. 17A, to perform the survival analysis 155, a user may select different analysis parameters 1702, comprising a feature 1704 or a diversity index 1706. As an example and not by way of limitation, the diversity index 1706 may comprise Shannon, Simpson, inverse Simpson, richness, etc. The user may then split into two or three groups 1708 to calculate the survival curves. The user may further select the progression-free survival (PFS)/overall survival (OS) phenotype 1710 and the event phenotype 1712. As an example and not by way of limitation, the PFS/OS phenotype 1710 may comprise phenotypic information relating to the patient or to the sample. As another example and not by way of limitation, the event phenotype 1712 may comprise phenotypic information relating to the patient or to the sample.

In particular embodiments, the survival analysis 155 may be based on a Kaplan-Meier curve. Kaplan-Meier is a traditional way for survival analysis 155, or timed events. FIG. 17B illustrates another example user interface for survival analysis. As illustrated in FIG. 17B, the user may have selected Prevotella as the feature 1704 and three groups. The user may have also selected PFS as the PFS/OS phenotype 1710 and PFS.CNSR2 as the event phenotype 1712. Accordingly, the survival analysis 155 showing the survival curves 1714 may map how much Prevotella these various groups had in their systems at different points in time. In FIG. 17B, Time 0 may be defined as time from randomization. Then the survival analysis 155 may start in the top left where we have three groups. All of them may start at 1, i.e., a 100% survival. As time progresses to 0, 2, 4, 6, and 8 months, more and more samples had an event. As an example and not by way of limitation, at month 8 the grey group may have 65% survived whereas the black and the red group may have only 25% survived. By choosing a particular individual, a user may see a line of the abundance jumping from one to the other. The user may also compare categorical analysis. As an example and not by way of limitation, the user may compare different races or different countries.

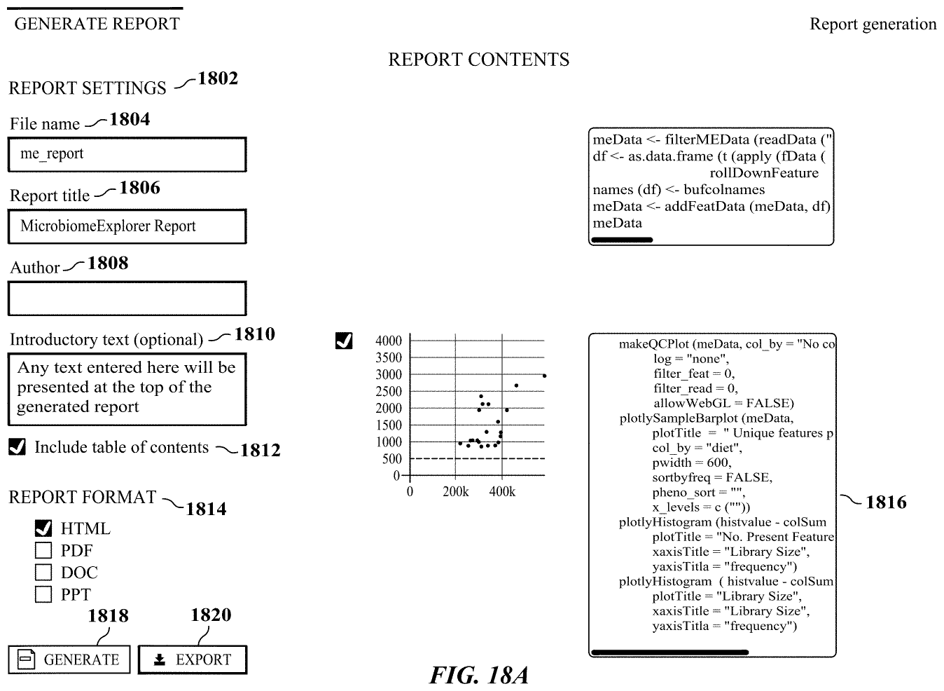

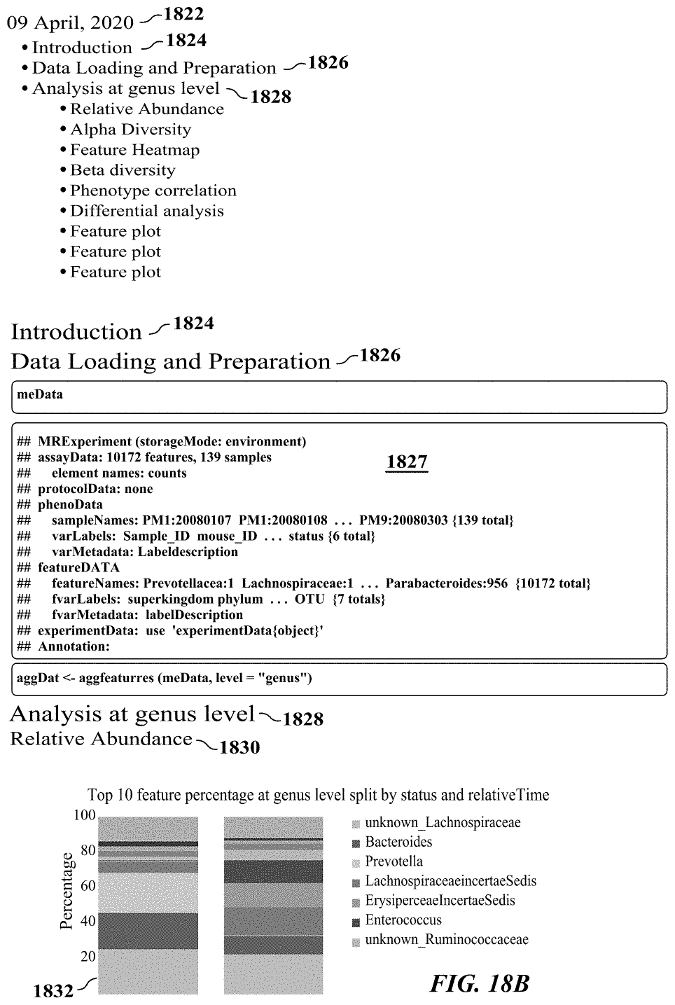

Once an analysis is complete, a user may share the results with other users or download the results for further analysis. In particular embodiments, the software tool 120 may enable the user to download the results by providing the option to include any part or all of the analysis in a report. In particular embodiments, the exportable report may be generated based on one or more report setting specified by a user via the software tool 120. The one or more report settings may comprise one or more of a file name, a report title, author information, or introductory text. In particular embodiments, the exportable report may be in a format specified by the user. The software code may be identified as related to a particular analysis of the microbiome analyses. In particular embodiments, the report may be fully reproducible outside of the software tool 120. FIG. 18A illustrates an example user interface for report generation via the software tool 120. In the report settings 1802, the user may choose the file name 1804, add a report title 1806, select authors 1808, and optionally add any other introductory text 1810 to be included in the outputted report. The user may also determine whether to include table of contents 1812. In particular embodiments, four different output report formats 1814 may be available by default. As an example and not by way of limitation, these report formats 1814 may comprise HTML, PDF, DOC and PPT. In particular embodiments, the report generation 160 may rely on the availability of external programs. The available output formats may be restricted in global.R. In addition to the basic report settings 1802, the user may also review any analysis made and choose which parts should be included in the report and which should not be included. For each analysis element, the relevant R code may be shown next to an icon illustrating the type of analysis, e.g., code 1816 illustrated in FIG. 18A. Showing the relevant code may help users without a computational background to identify which section is related to which part of their analysis. In particular embodiments, steps that are essential such as data loading or aggregation may not be deselected.

In order to obtain the report, the user may first click the “Generate” 1818 button. Then the relevant R code collected during the analysis anytime the user clicks the “Export” 1820 button may be written to a temporary file. The temporary R file may be then knitted into a document. As an example and not by way of limitation, the document may be a R markdown (.rmd) file. As another example and not by way of limitation, knitting the temporary R file into the document may be based on knitr::spin. In particular embodiments, the R markdown document may be subsequently rendered to the desired output format(s), e.g., with rmarkdown::render. R code chunks may be enhanced with basic parameters to optimize the sizing of figures.

FIG. 18B illustrates an example report generated by the software tool 120. As indicated in FIG. 18B, the report may include the time 1822. The report may comprise different sections, including introduction 1824, data loading and preparation 1826, and analysis at genus level 1828. The analysis at genus level 1828 may further comprise several sub-sections such as relative abundance, alpha diversity, feature heatmap, beta diversity, phenotype correlation, differential analysis, and different feature plots. The user may click on each section or sub-section to see more details of that section. As an example and not by way of limitation, for data loading and preparation 1826, the report may show the details of the data in the text box 1827. As another example and not by way of limitation, the relative abundance 1830 of the analysis at genus level may display a bar plot 1832 showing the top 10 feature percentage at genus level split by status and relativeTime.

During the process of report generation, any analysis may be repeated for each output format while calling the render function. In particular embodiments, the software tool 120 may generate reports as a background process and send the results to users in different communication methods. As an example and not by way of limitation, the results may be sent to users via email. In particular embodiments, once the render process is completed, the user may download the results by clicking “EXPORT” 1820. As an example and not by way of limitation, the user may then obtain a zip folder which holds both the R markdown document as well as any output formats specified by the user. In particular embodiments, the R markdown document may be edited and re-rendered outside of the software tool. The user may need to adjust the path to the input data to do so. In particular embodiments, all functionality available via the software tool may be also accessible via the command line in a standard R environment.

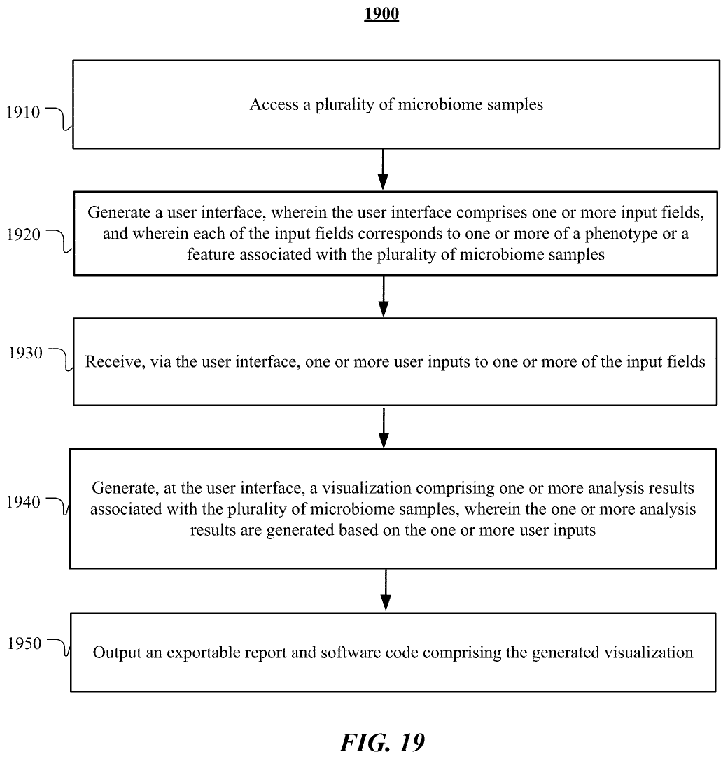

FIG. 19 illustrates an example diagram flow 1900 using the software tool 120 for microbiome analyses. The diagram flow may begin at step 1910, where the software tool 120 may access a plurality of microbiome samples. At step 1920, the software tool 120 may generate a user interface, wherein the user interface comprises one or more input fields, and wherein each of the input fields corresponds to one or more of a phenotype or a feature associated with the plurality of microbiome samples. At step 1930, the software tool 120 may receive, via the user interface, one or more user inputs to one or more of the input fields. At step 1940, the software tool 120 may generate, at the user interface, a visualization comprising one or more analysis results associated with the plurality of microbiome samples, wherein the one or more analysis results are generated based on the one or more user inputs. At step 1950, the software tool 120 may output an exportable report and software code comprising the generated visualization. Particular embodiments may repeat one or more steps of the method of FIG. 19, where appropriate. Although this disclosure describes and illustrates particular steps of the diagram flow of FIG. 19 as occurring in a particular order, this disclosure contemplates any suitable steps of the diagram flow of FIG. 19 occurring in any suitable order. Moreover, although this disclosure describes and illustrates an example method for suggesting candidate gestures for auto-completion, including the particular steps of the diagram flow of FIG. 19, this disclosure contemplates any suitable diagram flow for using the software tool for microbiome analyses, including any suitable steps, which may include all, some, or none of the steps of the diagram flow of FIG. 19, where appropriate. Furthermore, although this disclosure describes and illustrates particular components, devices, or systems carrying out particular steps of the diagram flow of FIG. 19, this disclosure contemplates any suitable combination of any suitable components, devices, or systems carrying out any suitable steps of the diagram flow of FIG. 19.

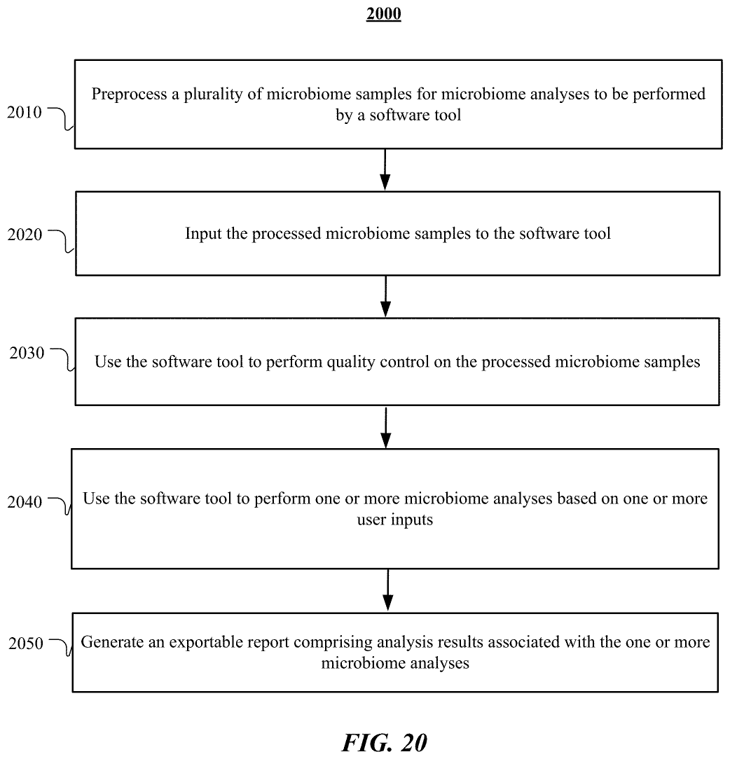

FIG. 20 illustrates an example method 2000 for microbiome analyses. The method may begin at step 2010, where a computing system may preprocess a plurality of microbiome samples for microbiome analyses to be performed by a software tool 120. At step 2020, the computing system may input the processed microbiome samples to the software tool 120. At step 2030, the computing system may use the software tool 120 to perform quality control on the processed microbiome samples. At step 2040, the computing system may use the software tool 120 to perform one or more microbiome analyses based on one or more user inputs. At step 2050, the computing system may generate an exportable report comprising analysis results associated with the one or more microbiome analyses. Particular embodiments may repeat one or more steps of the method of FIG. 20, where appropriate. Although this disclosure describes and illustrates particular steps of the method of FIG. 20 as occurring in a particular order, this disclosure contemplates any suitable steps of the method of FIG. 20 occurring in any suitable order. Moreover, although this disclosure describes and illustrates an example method for microbiome analyses, including the particular steps of the method of FIG. 20, this disclosure contemplates any suitable method for microbiome analyses, including any suitable steps, which may include all, some, or none of the steps of the method of FIG. 20, where appropriate. Furthermore, although this disclosure describes and illustrates particular components, devices, or systems carrying out particular steps of the method of FIG. 20, this disclosure contemplates any suitable combination of any suitable components, devices, or systems carrying out any suitable steps of the method of FIG. 20.

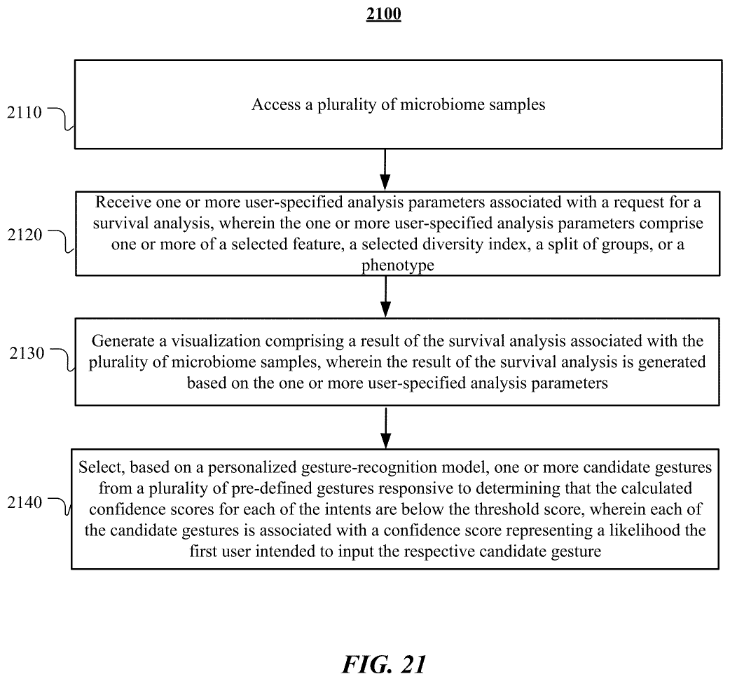

FIG. 21 illustrates an example method 2100 for survival analysis. The method may begin at step 2110, where a computing system may access a plurality of microbiome samples. At step 2120, the computing system may receive one or more user-specified analysis parameters associated with a request for a survival analysis 155, wherein the one or more user-specified analysis parameters comprise one or more of a selected feature, a selected diversity index, a split of groups, or a phenotype. At step 2130, the computing system may generate a visualization comprising a result of the survival analysis 155 associated with the plurality of microbiome samples, wherein the result of the survival analysis 155 is generated based on the one or more user-specified analysis parameters. At step 2140, the computing system may generate an exportable report and software code comprising the generated visualization. Particular embodiments may repeat one or more steps of the method of FIG. 21, where appropriate. Although this disclosure describes and illustrates particular steps of the method of FIG. 21 as occurring in a particular order, this disclosure contemplates any suitable steps of the method of FIG. 21 occurring in any suitable order. Moreover, although this disclosure describes and illustrates an example method for survival analysis, including the particular steps of the method of FIG. 21, this disclosure contemplates any suitable method for survival analysis, including any suitable steps, which may include all, some, or none of the steps of the method of FIG. 21, where appropriate. Furthermore, although this disclosure describes and illustrates particular components, devices, or systems carrying out particular steps of the method of FIG. 21, this disclosure contemplates any suitable combination of any suitable components, devices, or systems carrying out any suitable steps of the method of FIG. 21.

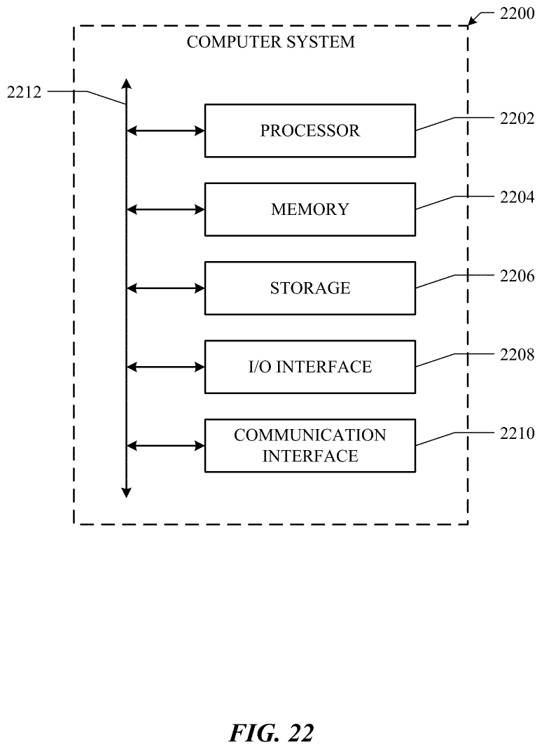

FIG. 22 illustrates an example computer system 2200. In particular embodiments, one or more computer systems 2200 perform one or more steps of one or more methods described or illustrated herein. In particular embodiments, one or more computer systems 2200 provide functionality described or illustrated herein. In particular embodiments, software running on one or more computer systems 2200 performs one or more steps of one or more methods described or illustrated herein or provides functionality described or illustrated herein. Particular embodiments include one or more portions of one or more computer systems 2200. Herein, reference to a computer system may encompass a computing device, and vice versa, where appropriate. Moreover, reference to a computer system may encompass one or more computer systems, where appropriate.

This disclosure contemplates any suitable number of computer systems 2200. This disclosure contemplates computer system 2200 taking any suitable physical form. As example and not by way of limitation, computer system 2200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 2200 may include one or more computer systems 2200; be unitary or distributed; span multiple locations; span multiple machines; span multiple data centers; or reside in a cloud, which may include one or more cloud components in one or more networks. Where appropriate, one or more computer systems 2200 may perform without substantial spatial or temporal limitation one or more steps of one or more methods described or illustrated herein. As an example and not by way of limitation, one or more computer systems 2200 may perform in real time or in batch mode one or more steps of one or more methods described or illustrated herein. One or more computer systems 2200 may perform at different times or at different locations one or more steps of one or more methods described or illustrated herein, where appropriate.

In particular embodiments, computer system 2200 includes a processor 2202, memory 2204, storage 2206, an input/output (I/O) interface 2208, a communication interface 2210, and a bus 2212. Although this disclosure describes and illustrates a particular computer system having a particular number of particular components in a particular arrangement, this disclosure contemplates any suitable computer system having any suitable number of any suitable components in any suitable arrangement.

In particular embodiments, processor 2202 includes hardware for executing instructions, such as those making up a computer program. As an example and not by way of limitation, to execute instructions, processor 2202 may retrieve (or fetch) the instructions from an internal register, an internal cache, memory 2204, or storage 2206; decode and execute them; and then write one or more results to an internal register, an internal cache, memory 2204, or storage 2206. In particular embodiments, processor 2202 may include one or more internal caches for data, instructions, or addresses. This disclosure contemplates processor 2202 including any suitable number of any suitable internal caches, where appropriate. As an example and not by way of limitation, processor 2202 may include one or more instruction caches, one or more data caches, and one or more translation lookaside buffers (TLBs). Instructions in the instruction caches may be copies of instructions in memory 2204 or storage 2206, and the instruction caches may speed up retrieval of those instructions by processor 2202. Data in the data caches may be copies of data in memory 2204 or storage 2206 for instructions executing at processor 2202 to operate on; the results of previous instructions executed at processor 2202 for access by subsequent instructions executing at processor 2202 or for writing to memory 2204 or storage 2206; or other suitable data. The data caches may speed up read or write operations by processor 2202. The TLBs may speed up virtual-address translation for processor 2202. In particular embodiments, processor 2202 may include one or more internal registers for data, instructions, or addresses. This disclosure contemplates processor 2202 including any suitable number of any suitable internal registers, where appropriate. Where appropriate, processor 2202 may include one or more arithmetic logic units (ALUs); be a multi-core processor; or include one or more processors 2202. Although this disclosure describes and illustrates a particular processor, this disclosure contemplates any suitable processor.

In particular embodiments, memory 2204 includes main memory for storing instructions for processor 2202 to execute or data for processor 2202 to operate on. As an example and not by way of limitation, computer system 2200 may load instructions from storage 2206 or another source (such as, for example, another computer system 2200) to memory 2204. Processor 2202 may then load the instructions from memory 2204 to an internal register or internal cache. To execute the instructions, processor 2202 may retrieve the instructions from the internal register or internal cache and decode them. During or after execution of the instructions, processor 2202 may write one or more results (which may be intermediate or final results) to the internal register or internal cache. Processor 2202 may then write one or more of those results to memory 2204. In particular embodiments, processor 2202 executes only instructions in one or more internal registers or internal caches or in memory 2204 (as opposed to storage 2206 or elsewhere) and operates only on data in one or more internal registers or internal caches or in memory 2204 (as opposed to storage 2206 or elsewhere). One or more memory buses (which may each include an address bus and a data bus) may couple processor 2202 to memory 2204. Bus 2212 may include one or more memory buses, as described below. In particular embodiments, one or more memory management units (MMUs) reside between processor 2202 and memory 2204 and facilitate accesses to memory 2204 requested by processor 2202. In particular embodiments, memory 2204 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 2204 may include one or more memories 2204, where appropriate. Although this disclosure describes and illustrates particular memory, this disclosure contemplates any suitable memory.

In particular embodiments, storage 2206 includes mass storage for data or instructions. As an example and not by way of limitation, storage 2206 may include a hard disk drive (HDD), a floppy disk drive, flash memory, an optical disc, a magneto-optical disc, magnetic tape, or a Universal Serial Bus (USB) drive or a combination of two or more of these. Storage 2206 may include removable or non-removable (or fixed) media, where appropriate. Storage 2206 may be internal or external to computer system 2200, where appropriate. In particular embodiments, storage 2206 is non-volatile, solid-state memory. In particular embodiments, storage 2206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 2206 taking any suitable physical form. Storage 2206 may include one or more storage control units facilitating communication between processor 2202 and storage 2206, where appropriate. Where appropriate, storage 2206 may include one or more storages 2206. Although this disclosure describes and illustrates particular storage, this disclosure contemplates any suitable storage.

In particular embodiments, I/O interface 2208 includes hardware, software, or both, providing one or more interfaces for communication between computer system 2200 and one or more I/O devices. Computer system 2200 may include one or more of these I/O devices, where appropriate. One or more of these I/O devices may enable communication between a person and computer system 2200. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 2208 for them. Where appropriate, I/O interface 2208 may include one or more device or software drivers enabling processor 2202 to drive one or more of these I/O devices. I/O interface 2208 may include one or more I/O interfaces 2208, where appropriate. Although this disclosure describes and illustrates a particular I/O interface, this disclosure contemplates any suitable I/O interface.

In particular embodiments, communication interface 2210 includes hardware, software, or both providing one or more interfaces for communication (such as, for example, packet-based communication) between computer system 2200 and one or more other computer systems 2200 or one or more networks. As an example and not by way of limitation, communication interface 2210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 2210 for it. As an example and not by way of limitation, computer system 2200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 2200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 2200 may include any suitable communication interface 2210 for any of these networks, where appropriate. Communication interface 2210 may include one or more communication interfaces 2210, where appropriate. Although this disclosure describes and illustrates a particular communication interface, this disclosure contemplates any suitable communication interface.

In particular embodiments, bus 2212 includes hardware, software, or both coupling components of computer system 2200 to each other. As an example and not by way of limitation, bus 2212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 2212 may include one or more buses 2212, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A software tool for performing microbiome analyses, comprising:
accessing, by the software tool, a plurality of microbiome samples;
generating, by the software tool, a user interface associated with the software tool, wherein the user interface comprises one or more input fields, and wherein each of the input fields corresponds to one or more of a phenotype or a feature associated with the plurality of microbiome samples;
receiving, via the user interface of the software tool, one or more user inputs to one or more of the input fields;
generating, by the software tool at the user interface, a visualization comprising one or more analysis results associated with the plurality of microbiome samples, wherein the one or more analysis results are generated based on the one or more user inputs; and
outputting, by the software tool, an exportable report and software code comprising the generated visualization.

2. The software tool of claim 1, further comprising:
generating, by the software tool, one or more quality-control plots based on the accessed microbiome samples;
receiving, via the user interface of the software tool, a data-filtering input based on one or more of minimum sample presence, minimum number of features, or minimum number of reads;
generating, by the software tool, a subset of the accessed microbiome samples based on the data-filtering input; and
updating, by the software tool, the one or more quality-control plots based on the subset of the accessed microbiome samples.

3. The software tool of claim 1, further comprising:
receiving, via the user interface of the software tool, a data-aggregation input specifying a feature level; and
aggregating, by the software tool, the accessed microbiome samples to the specified feature level.

4. The software tool of claim 1, wherein the microbiome analyses comprise an intra-sample analysis comprising investigating microbial composition within a microbiome sample or a group of microbiome samples.

5. The software tool of claim 4, wherein the one or more analysis results comprise one or more of relative abundance, feature abundance, or alpha diversity.

6. The software tool of claim 5, wherein the one or more analysis results comprise relative abundance, wherein the relative abundance comprises one or more abundant features in a bar plot generated based on one or more user-specified analysis parameters, wherein the one or more user-specified analysis parameters comprise one or more of a phenotype, a faceting manner, or a feature, and wherein the bar plot is modifiable based on one or more of a number of features to show, a switch between showing percentage or reads, or a plot width.

7. The software tool of claim 5, wherein the one or more analysis results comprise feature abundance, wherein the feature abundance comprises an individual abundance of a specific feature as a box plot or a categorical scatterplot generated based on one or more user-specified analysis parameters, wherein the one or more user-specified analysis parameters comprise one or more of a phenotype, a faceting manner, or a feature, wherein the box plot or categorical scatterplot is modifiable based on one or more of a switch between showing points or not showing points, a switch between showing log scale or not showing log scale, a switch between showing percentage or reads, or a plot width.

8. The software tool of claim 5, wherein the one or more analysis results comprise alpha diversity, wherein the alpha diversity comprises a measure of a complexity or a diversity within a particular microbiome sample as a box plot generated based on one or more user-specified analysis parameters, wherein the one or more user-specified analysis parameters comprise one or more of a phenotype, a faceting manner, or a feature, and wherein the box plot is modifiable based on one or more of an index, a coloring manner, or a plot width.

9. The software tool of claim 1, wherein the microbiome analyses comprise an inter-sample analysis, the inter-sample analysis comprising determining differences between microbiome samples or a group of microbiome samples.

10. The software tool of claim 9, wherein the one or more analysis results comprise one or more of beta diversity or feature heatmap.

11. The software tool of claim 10, wherein the one or more analysis results comprise beta diversity, wherein the beta diversity comprises a measure of a complexity of communities between microbiome samples based on one or more user-specified analysis parameters, wherein the one or more user-specified analysis parameters comprise one or more of a distance matrix, an Adonis variable, or an Adonis strata, wherein the beta diversity is illustrated as a scatter plot generated based on principle component analysis, and wherein the scatter plot is modifiable based on one or more of a selection of one or more principal components, a coloring ellipse based on a phenotype, a shape based on a phenotype, a point size, or a plot width.

12. The software tool of claim 10, wherein the one or more analysis results comprise feature heatmap, wherein the feature heatmap comprises a visualization on differences and similarities between microbiome samples, wherein the feature heatmap is generated based on one or more of a number of top features sorted by a user-defined criteria or a user selected feature, and wherein the user-defined criteria comprises one or more of variance, Fano factor, or median absolute deviation.

13. The software tool of claim 1, wherein the microbiome analyses comprise a correlation analysis, the correlation analysis comprising a visualization of relationship between two features or a feature and a phenotype in a scatter plot, wherein the correlation analysis is generated based on an association-evaluation method comprising one or more of Spearman, Person, or Kendall, and wherein the correlation analysis is further generated based on one or more user-specified analysis parameters comprising one or more of a base feature, a correlation feature, or a correlation phenotype.

14. The software tool of claim 1, wherein the microbiome analyses comprise a differential abundance analysis, the differential abundance analysis comprising a test of null hypothesis that a mean or mean ranks between groups of microbiome samples are the same for a specific feature, wherein the differential abundance analysis is generated based on one or more user-specified analysis parameters comprising one or more of a testing method, a comparison phenotype, or a comparison level, and wherein the software tool further generates an interactive table for the differential abundance analysis, the interactive table being operable for a user to open feature plots showing specific levels selected by the user.

15. The software tool of claim 1, wherein the microbiome analyses comprise a longitudinal analysis, the longitudinal analysis comprising a comparison of microbial composition across time points or conditions, wherein the longitudinal analysis is generated based on one or more user-specified analysis parameters comprising one or more of a selected feature, a longitudinal phenotype, a phenotype level order, or a phenotype identifier, wherein the software tool further generates an interactive visualization of a feature plot corresponding to the longitudinal analysis, the interactive visualization being operable for a user to select and color one or more specific phenotype identifiers within the feature plot.

16. The software tool of claim 1, wherein the exportable report is generated based on one or more report setting specified by a user, wherein the one or more report settings comprise one or more of a file name, a report title, author information, or introductory text, wherein the exportable report is in a format specified by the user, and wherein the software code is identified as related to a particular analysis of the microbiome analyses.

17. One or more computer-readable non-transitory storage media embodying software that is operable when executed to:
access a plurality of microbiome samples;
generate a user interface, wherein the user interface comprises one or more input fields, and wherein each of the input fields corresponds to one or more of a phenotype or a feature associated with the plurality of microbiome samples;
receive, via the user interface, one or more user inputs to one or more of the input fields;
generate, at the user interface, a visualization comprising one or more analysis results associated with the plurality of microbiome samples, wherein the one or more analysis results are generated based on the one or more user inputs; and
output an exportable report and software code comprising the generated visualization.

18. A system comprising: one or more processors; and a non-transitory memory coupled to the processors comprising instructions executable by the processors, the processors operable when executing the instructions to:
access a plurality of microbiome samples;
generate a user interface, wherein the user interface comprises one or more input fields, and wherein each of the input fields corresponds to one or more of a phenotype or a feature associated with the plurality of microbiome samples;
receive, via the user interface, one or more user inputs to one or more of the input fields;
generate, at the user interface, a visualization comprising one or more analysis results associated with the plurality of microbiome samples, wherein the one or more analysis results are generated based on the one or more user inputs; and
output an exportable report and software code comprising the generated visualization.

19. A method comprising, by one or more computing systems:
preprocessing a plurality of microbiome samples for microbiome analyses to be performed by a software tool;
inputting the processed microbiome samples to the software tool;
using the software tool to perform quality control on the processed microbiome samples;
using the software tool to perform one or more microbiome analyses based on one or more user inputs; and
generating an exportable report comprising analysis results associated with the one or more microbiome analyses.

20. A method comprising, by one or more computing systems:
accessing a plurality of microbiome samples;
receiving one or more user-specified analysis parameters associated with a request for a survival analysis, wherein the one or more user-specified analysis parameters comprise one or more of a selected feature, a selected diversity index, a split of groups, or a phenotype;
generating a visualization comprising a result of the survival analysis associated with the plurality of microbiome samples, wherein the result of the survival analysis is generated based on the one or more user-specified analysis parameters; and
generating an exportable report and software code comprising the generated visualization.
